(19) United States
(12) Patent Application Publication
(10) Pub. No.: US 2016/0137712 A1
(43) Pub. Date: May 19, 2016

(54) Fusion Proteins With Dual Receptor Agonist Activities

(71) Applicant: ASKGENE PHARMA, INC., Camarillo, CA (US)

(72) Inventors: Yuefeng Lu, Newbury Park, CA (US); Jian-Feng Lu, Oak Park, CA (US); Aijun Wang, Camarillo, CA (US)

(73) Assignee: ASKGENE PHARMA, INC., Camarillo, CA (US)

(21) Appl. No.: 14/941,529

(22) Filed: Nov. 13, 2015

Related U.S. Application Data
(60) Provisional application No. 62/079,518, filed on Nov. 13, 2014.

(51) Int. Cl. C07K 14/605; C07K 14/575

(52) U.S. Cl. CPC C07K 14/605; C07K 14/575; C07K 2319/30; A61K 38/00

(57) ABSTRACT

The present disclosure relates to heterodimeric fusion proteins comprising two polypeptides, the first polypeptide comprising a first peptide (P1), a linker (L1), and a Fc region (F1), the second polypeptide comprising a second peptide (P2), a linker (L2), and an Fc region (F2), wherein P1 and P2 are each independently selected from GLP-1, GLP-1 analogues, glucagon, glucacon analogues, GIP, GIP analogues, oxyntomodulin, oxyntomodulin analogues, exendin and exendin analogues; wherein F is selected from an IgG Fc, an IgA Fc, an IgE Fc, an IgGM Fc, and their analogues; wherein the C-terminals of the peptides are linked, though the Linker L, to the N-terminals of the Fc region F. In one embodiment, the fusion proteins disclosed herein have agonist activity against at least two of the GLP-1 receptor, the GIP receptor, and the glucagon receptor.

### CROSS-REFERENCES TO RELATED APPLICATIONS

This patent application claims the right of priority pursuant to 35 U.S.C. §119(e) and is entitled to the benefit of the filing date of U.S. Provisional Patent Application 62/079,518, filed on Nov. 13, 2014, the content of which is hereby expressly incorporated by reference in its entirety.

### REFERENCE TO SEQUENCE LISTING

The entire content of the following electronic submission of the sequence listing via the USPTO EFS-WEB server, as authorized and set forth in MPEP §1730 II.B.2(a)(C), is hereby expressly incorporated by reference in its entirety for all purposes. The sequence listing is identified on the electronically filed text file as follows: File Name: 3IPAG3-0004USSeqList_ST25; Date of Creation: Nov. 13, 2015; Size (bytes): 100 KB.

### INTRODUCTION

It is estimated that there are over 370 million patients with diabetes worldwide, with approximately 90% of them having type 2 diabetes. In general, Type 2 diabetics still make insulin, but the insulin cannot be used effectively by the body's cells. This is primarily because the amount of insulin produced in response to rising blood sugar levels is not sufficient to allow cells to efficiently take up glucose and thus, reduce blood sugar levels.

Pre-proglucagon is a 158 amino acid precursor polypeptide (see, e.g., sp|P01275|GLUC_HUMAN Glucagon, accessed Nov. 10, 2014, the reference sequence including the 20 amino acid signal peptide, for a 178 amino acid peptide) that is processed in different tissues to form a number of different proglucagon-derived peptides, including glucagon, glucagon-like peptide-1 (GLP-1), glucagon-like peptide-2 (GLP-2) and oxyntomodulin (OXM). These peptides are involved in a wide variety of physiological functions, including glucose homeostasis, insulin secretion, gastric emptying, and intestinal growth, as well as the regulation of food intake. Glucagon is a 29-amino acid peptide that corresponds to amino acids 33 through 61 of pre-proglucagon, while GLP-1 is produced as a 37-amino acid peptide that corresponds to amino acids 72 through 108 of pre-proglucagon. GLP-1(7-36) amide or GLP-1(7-37) acid are biologically potent forms of GLP-1, that demonstrate essentially equivalent activity at the GLP-1 receptor.

GLP-1 plays important roles in regulating insulin secretion, blood glucose level and metabolism. Unfortunately, it has very short half-life in circulation due to DP IV degradation and its small molecular weight. Several versions of GLP-1 analogues have been developed to retain GLP-1 activities while also have various degree of resistance to DP-IV degradation. Additional modifications such as GLP-1 analogue conjugates and fusion proteins significantly increased their half-lives in vivo. Those modifications include pegylation, GLP-1 analogue-albumin fusion proteins, and GLP-1 analogue-Fc fusion proteins. Because GLP-1 products are used for treating diabetics, which is a chronic disease, minimizing immunogenicity is also very important.

Several GLP-1 analogs have been successfully launched into the market. Among them are VICTOZA™ (Novo Nordisc) and BYETTA™ (Bristol Myers Squib). A number of GLP-1 analogs are in various stage of clinical development. One of the noticeable ones is DULAGLUTIDE™ (Eli Lilly), which has successfully completed phase 3 clinical development with outstanding efficacy data comparing to its peers. DULAGLUTIDE™ is a GLP-1 analogue-Fc fusion protein. As disclosed in patent EP 1641823, its GLP-1 analogue domain contained a substitution at the eighth amino acid to Glycine (i.e., “Gly(8)”) in order to obtain resistance against dipeptidyl-peptidase IV (DP-IV) degradation. Its Fc domain is an analogue of IgG4 Fc. Several modifications were made to the fusion protein in order to minimize or deplete Fc functionality and to reduce potential immunogenicity.

Glucose-dependent insulinotrophic polypeptide (aka gastric inhibitory polypeptide, hereinafter “GIP”) is a 42 amino acid peptide having the sequence of >sp|P09681|amino acids 52-93, as accessed on Nov. 9, 2014. Like GLP-1, GIP can induce glucose-dependent pancreatic beta-cell insulin secretion. GIP also has extra-pancreatic activities which further promote glucose lowering. While there are similarities, GLP-1 and GIP function through two different mechanisms. GIP analogs with a substitution at the second amino acid to glycine or serine (i.e., “Gly(2)” or “Ser(2)”) had increased resistance to DP-IV degradation (Gault, et al., Improved biological activity of Gly2- and Ser2-substituted analogues of glucose-dependent insulinotrophic polypeptide, J Endocrinol. 176(1): 133-141, 2003).

Glucagon is a peptide hormone secreted by the pancreas, which raises blood glucose levels. The pancreas releases glucagon when blood glucose levels fall too low. Glucagon causes the liver to convert stored glycogen into glucose, which is released into the bloodstream. High blood glucose levels stimulate the release of insulin. Insulin allows glucose to be taken up and used by insulin-dependent tissues. Thus, glucagon and insulin are part of a feedback system that keeps blood glucose levels at a stable level.

GLP-1 analogs showed limited weight loss activities in patients. Dual activity agonists for GLP-1, GIP and/or glucagon receptors showed superior efficacy in treating diabetes and related illness, especially weight loss. For example, it was reported that a peptide with potent, balanced co-agonism at both GLP-1/GIP receptors showed synergistic and superior efficacy in animal and human studies (Finan, et al., Unimolecular dual incretins maximize metabolic benefits in rodents, monkeys, and humans, Sci. Transl. Med. 5(209): 209ra151, 2013). For another example, dual GLP-1/glucagon receptor (GLP1R/GCGR) agonist showed significant weight loss without evidence of hyperglycemia in animal study (Day, et al., A new glucagon and GLP-1 co-agonist eliminates obesity in rodents, Nat. Chem. Biol. (10): 749-757, 2009). Oxyntomodulin has dual agonist activity for both GLP-1 and glucagon receptors, though the activities were lower comparing to GLP-1 and glucagon. Based on the dual function of the oxyntomodulin peptide, it has been actively studied as a drug for the treatment of obesity. However, it has been reported that the anti-obesity drug including oxyntomodulin has a short in vivo half-life and weak therapeutic efficacy, even though administered at a high dose three times a day. Thus, many efforts have been made to improve the in vivo half-life or therapeutic effect of oxyntomodulin on obesity. For example, Korean Patent 925017 discloses a pharmaceutical composition including oxyntomodulin as an active ingredient for the treatment of overweight human, which is administered via an oral, parenteral, mucosal, rectal, subcutaneous, or transdermal route.

A number of peptides possessing dual agonist activities for GLP-1/GIP receptors or GLP-1/Glucagon receptors (US Patent Application Publication 2013/0090286) in a single peptide. Those peptides are often chemically synthesized and modified with pegylation or attached with fatty acid chains in order to increase their half-lives in vivo. Those peptides can also be fused or conjugated to the Fc fragment to create peptide-Fc fusion proteins to enhance their activities through dimerization and improve their in vivo half-lives through the Fc domain.

IgG Fc may be used as carrier to extend in vivo half-lives of therapeutic peptides such as GLP-1 analogues. IgG Fc fusion proteins has in vivo half-lives ranged from a few days to over a week (Aavang Tibble, et al., Longer Acting GLP-1 Receptor Agonists and the Potential for Improved Cardiovascular Outcomes, A Review of Current Literature, Expert Rev. Endocrinol. Metab. 8(3): 247-259, 2013) Meanwhile, full antibody molecules may also be used as carrier for therapeutic peptides. Antibody molecules can have in vivo half-lives over 3 weeks (Hinton, et al., An engineered human IgG1 antibody with longer serum half-life, J. Immunol. 176(1): 346-356, 2006). In addition, modifications have been made to the Fc domain to further extend its half-life in vivo. For example, introduction of mutations T250Q/M428L to an IgG1 molecule led to a significant increase in the serum half-life of the IgG1 molecule, as disclosed in U.S. Pat. No. 7,217,798, the content of which is hereby expressly incorporated by reference in its entirety for all purposes. Other mutations have also been made to increase the binding affinity of Fc to FcRN and extend the serum half-life, such as disclosed in U.S. Pat. No. 8,394,925, the content of which is hereby expressly incorporated by reference in its entirety for all purposes.

Fc fusion proteins are often homo-dimers. However, hetero-dimers can also be constructed. For example, U.S. Pat. No. 7,642,228, the content of which is hereby expressly incorporated by reference in its entirety for all purposes, disclosed that heterodimers of antibodies and Fc-fusion proteins can be constructed using the “Knob-and-Hole” concept.

The fusion proteins can be made through recombinant technology. Both prokaryotic and eukaryotic systems may be used to express recombinant proteins. Recombinant Fc fusion proteins produced by eukaryotic systems are often glycosylate and certain types such as IgG1 molecules may also have Fc functionalities. In addition, molecules expressed in mammalian systems such as CHO are often secreted to the culture media. Signal peptides are often used and processed, and there are usually no methionine at the N-terminal.

Recombinant fusion proteins expressed by prokaryotic systems such as E. coli do not have glycosylation. For the ones expressed in E. coli, the fusion proteins are also frequently produced in inclusion bodies, which need to be refolded to become active molecules. In addition, a methionine residue is added to the N-terminals of the proteins. Depending on the N-terminal amino acid of the fusion protein, the added methionine may or may not be processed. Methionine Amino Peptidase (MetAP) is able to cleave N-terminal methionine if the second amino acid has a small side chain, such as glycine. Co-transformation of a mutated MetAP would reduce the specificity of the protease. For example, engineered E. coli MetAP with triple mutations Y168G, M206T and Q233G was able to remove the N-terminal methionine from bulky amino acids such as tryptophan and histidine (Liao, et al., Removal of N-terminal methionine from recombinant proteins by engineered E. coli methionine aminopeptidase, Protein Sci. 13(7): 1802-1810, 2004).

Unnatural amino acid(s) may be introduced into peptides, if chemically synthesized. For example, unnatural amino acid aminoisobutyric acid (Aib) has been introduced into GLP-1 analogue peptides. GLP-1 analogue containing Gly(8) has enhanced resistance to DP-IV enzyme degradation comparing to the native GLP-1 peptide. However, further optimization may also be possible. It was shown that, during in vitro porcine plasma study, Gly(8) analogue had a half-life of 159 min, vs Aib(8) analogue remained intact after 6 hours (Deacon, et al., Dipeptidyl peptidase IV resistant analogues of glucagon-like peptide-1 which have extended metabolic stability and improved biological activity, Diabetologia. 41(3): 271-278, 1998). Aib(8) analogue also showed higher GLP-1 receptor binding affinity (IC50; Aib8=0.45 nmol/l; Gly8=2.8 nmol/l). In the same report, it further showed that, in the activity study with isolated pancreas, Aib(8) analogue showed higher efficacy than Gly(8) analogue. Therefore, unnatural amino acid can be introduced into GLP-1 peptide in order to further enhance the performance of the GLP-1 analogues.

Gly(8) GLP-1 analogue-Fc fusion protein can be produced entirely through recombinant technology. It may not be feasible to introduce a substitution at the eighth amino acid of GLP-1 to Aib (i.e., “Aib(8)”) or a D-amino acid (e.g. D-Ala) into the GLP-1 analogue fusion protein through standard recombinant technology. Aib(8) or D-amino acid can be introduced into the peptide through peptide synthesis. The chemically synthesized peptide can be chemically conjugated to a carrier such as albumin or Fc, such as disclosed in patent application WO2012173422; however the sites of conjugation on the Fc appeared to be not specific and the ratio of peptide to Fc may also be variable, potentially resulting into significant heterogeneity of the conjugated molecules. Site-specific conjugation and native chemical ligation (NCL) may be used to generate GLP-1 analogue-Fc fusion protein with high homogeneity.

Two different peptides, e.g. a GLP-1 analogue and a glucagon analogue, or a GLP-1 analogue and a GIP analogue, of desirable efficacies can be fused/conjugated to a same carrier such as an antibody molecule or an Fc fragment. Having two independent peptides fused/conjugated to the antibody molecule or an Fc fragment allows additional flexibility in selecting the more desirable peptides and balanced activities for each of the targeted receptors. A combination of recombinant expression, chemical native ligation and/or site-specific chemical conjugation can be applied to produce fusion proteins with these dual or multiple receptor agonist activities.

Finan et al (2013) of Roche reported that GLP-1/GIP receptor dual agonist peptide enhanced the therapeutic effects more than GLP-1 receptor agonist alone. Several companies, including Merck, Zealand Pharmaceuticals, and Hanmi Science Co, have carried out studies (Pocai, Unraveling oxyntomodulin, GLP1's enigmatic brother, J. Endocrinol. 215: 335-346, 2012), WO2012173422), which showed therapeutic effects of oxyntomodulin analogs, which are GLP-1/Glucagon receptor co-agonist. Other unimolecule versions of GLP-1/Glucagon receptor co-agonists have also been disclosed previously. ZP2495 showed increased cardiac performance in insulin-resistant hearts (Axelsen, et al., Glucagon and A Glucagon-GLP-1 Dual-Agonist Increases Cardiac Performance with Different Metabolic Effects in Insulin-Resistant Hearts, Br. J. Pharmacol., 165: 2736-2748, 2012). DiMarchi and colleagues showed that unimolecule glucagon and GLP-1 co-agonist eliminated obesity in rodents (Day, et al., A new glucagon and GLP-1 co-agonist eliminates obesity in rodents, Nat. Chem. Biol. 10: 749-757, 2009).

Oxyntomodulin analogues have been modified to extend their half-lives in circulation, through acylation (Druce, et al., Investigation of structure-activity relationships of Oxyntomodulin (Oxm) using Oxm analogs, Endocrinol. 150(4): 1712-1722, 2009), conjugation with Fc domain (WO 2012/173422), and pegylation (e.g. Zp2929). Oxyntomodulin and its analogues have significantly reduced activities for the glucagon and GLP-1 receptors. Most of the other unimolecular co-agonists referenced above were chemically synthesized and pegylated to extend their half-lives in vivo. They were typically monomers.

Functional dimers, e.g., by fusion to Fc, could significantly enhance the activity of the co-agonists discussed above. In the case of GLP-1 analogues, DURAGLUTIDE™ from Lilly is a GLP-1 analogue fused to IgG4 Fc, where the two GLP-1 analogues in each molecule are in parallel and a functional dimer; while ALBUGLUTIDE™ from GSK is a GLP-1-human serum albumin (HSA) fusion protein, where the two GLP-1 analogues in each molecule is in tandem. The right N-terminal is important for the activity for GLP-1 and its analogues, which likely decreases the activity of the 2^{nd} GLP-1 analogue in the tandem of ALBUGLUTIDE™. Even with that, it's surprising to see the dosage difference between DURAGLUTIDE™ (up to 1.5 mg/dose) vs ALBUGLUTIDE™ (30-50 mg per dose). This significantly higher efficacy (or lower dosage) of DURAGLUTIDE™ is consistent with the effect of the functional dimerization of GLP-1 analogue.

As opposed to the compositions described above, the present specification contemplates the use of a dual receptor agonist/antagonist/inhibitor. The efficacy obtained with a dual receptor agonist/antagonist/inhibitor of the present disclosure is synergistically improved in comparison to either the native protein agonist/antagonist/inhibitor or a construct directed to a single receptor.

### SUMMARY

Aspects of the present specification disclose fusion proteins comprising two polypeptides. The fusion proteins disclosed herein can comprise a first polypeptide comprising a first peptide (P1) a linker (L1) and an Fc region (F1) and a second polypeptide comprising a second peptide (P2), a linker (L2) and a Fc region (F2). The disclosed P1 and P2 peptides are each independently a GLP-1, a GLP-1 analogue, a glucagon, a glucacon analogue, a GIP, a GIP analogue, an oxyntomodulin, an oxyntomodulin analogue, an exendin or an exendin analogue. The disclosed F1 and F2 Fc regions are each independently an IgG Fc, an IgA Fc, an IgM Fc, an IgD Fc, an IgE Fc, SEQ ID NO: 35 or an analogue thereof. The C-terminals of the P1 and P2 peptides are linked, though the Linkers L1 and L2, to the N-terminals of the F1 and F2 Fc regions. The fusion proteins disclosed herein can be dimers, such as a homodimer and a heterodimer.

Other aspects of the present specification disclose formulations comprising the fusion proteins disclosed herein as well as pharmaceutical composition comprising the fusion proteins disclosed herein.

Other aspects of the present specification disclose a method of treating disorder by administering a fusion protein disclosed herein to an individual in need of such treatment. A disorder disclosed herein includes diabetes, obesity, inducement of weight loss in an overweight individual or steatosis.

Other aspects of the present specification disclose a fusion protein disclosed herein for use in treating a disorder as well as a use of a fusion proteins disclosed herein for treating a disorder. A disorder disclosed herein includes diabetes, obesity, inducement of weight loss in an overweight individual or steatosis.

Other aspects of the present specification disclose a use of a fusion proteins disclosed herein in the manufacture of a medicament for the treatment of a disorder. A disorder disclosed herein includes diabetes, obesity, inducement of weight loss in an overweight individual or steatosis.

### BRIEF DESCRIPTION OF THE DRAWINGS

FIG. 1 shows a schematic drawings of peptide-Fc fusion proteins. The peptides are fused, through a linker, to the N-terminals of the Fc fragment. The linker has an amino acid sequence (GGGGS)ₙ, wherein n=2, 3 or 4 (SEQ ID NO: 1).

FIG. 2 shows a schematic drawings of peptide-Fc fusion proteins where one of the peptides contains an unnatural amino acid (*). The peptides are fused or conjugated, through a linker, to the N-terminals of the Fc fragment. The linker has an amino acid sequence (GGGGS)ₙ, wherein n=2, 3 or 4 (SEQ ID NO:1) with or without additional modifications.

FIG. 3 shows a schematic drawings of peptide-Fc fusion proteins where each of the peptides contains an unnatural amino acid (*). The peptides are fused or conjugated, through a linker, to the N-terminals of the Fc fragment. The linker has an amino acid sequence (GGGGS)ₙ, wherein n=2, 3 or 4 (SEQ ID NO:1) with or without additional modifications.

FIG. 4 shows a schematic drawings of native chemical ligation reaction. In the native chemical ligation reaction, the thiolate group of the N-terminal cysteine residue of Unprotected Peptide 2 (e.g. an IgG4 Fc analogue) attacks the C-terminal thioester of Unprotected Peptide 1 (e.g. a GLP-1 analogue). This leads to form a thioester intermediate 3, which rearranges and results in the formation of a native amide (‘peptide’) bond 4 at the ligation site to form the fusion protein.

FIG. 5 shows a graph of an activity assay based on cAMP induction.

### DETAILED DESCRIPTION

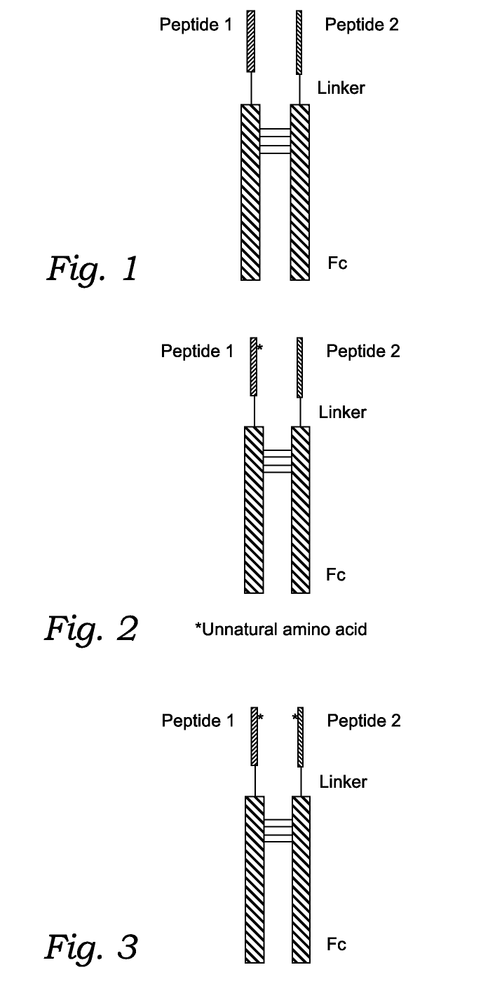

Disclosed here is an open platform containing two peptides, with each peptide fused to the N-terminals of a carrier molecule. This open platform allows flexibility in selecting each of the peptides, potentially with additional modifications, in order to reach the ideal ratio for the activities for the concerned receptors, and to achieve synergistic therapeutic effects. The peptides include a GLP-1, an exendin, a GIP, an oxyntomodulin, a Glugagon, and analogs of each of the peptide mentioned above. The carrier molecule includes a Fc fragment, such as an IgG Fc fragment (including an IgG1, an IgG2, and/or an IgG4), an IgA Fc fragment, an IgE Fc fragment, an IgM Fc fragment, and their analogues. The peptides are fused to the N-terminals of the carrier molecule through a peptide linker. This platform allows constructions of the following two classes of fusion proteins with dual agonist activities: a) Peptide-Fc fusion proteins (FIG. 1); and b) Peptide-Fc fusion proteins wherein one or both peptides contain at least one unnatural amino acid (FIGS. 2 and 3).

The fusion proteins can be made entirely through recombinant expression; alternatively, it can be made through protein native chemical ligation (NCL) or site specific conjugation, wherein the peptide is chemically synthesized and the carrier molecule is recombinant expressed. The resulted molecules possess dual-receptor co-agonist activities and in certain cases tri-receptor co-agonists. The disclosure fusion proteins have therapeutic effects for obesity, diabetes and related conditions.

The present specification discloses fusion proteins. In one embodiment, a fusion protein disclosed herein is a homodimeric fusion protein comprising two polypeptides, the first polypeptide comprising a peptide (P1) a linker (L1) and an Fc region (F1) and the second polypeptide comprising a peptide (P1), a linker (L2) and an Fc region (F2). In an aspect of this embodiment, P1 includes a GLP-1, a GLP-1 analogue, a glucagon, a glucacon analogue, a GIP, a GIP analogue, an oxyntomodulin, an oxyntomodulin analogue, an exendin or an exendin analogue. In another aspect of this embodiment, F1 and F2 each independently include an IgG Fc, an IgA Fc, an IgE Fc, an IgGM Fc, or an analogue thereof. In yet another aspect of this embodiment, the C-terminals of peptides P1 and P2 are linked, though the Linkers L1 and L2, to the N-terminals of the Fc regions F1 and F2.

In another embodiment, a fusion protein disclosed herein is a heterodimeric fusion protein comprising two polypeptides, the first polypeptide comprising a first peptide (P1) a linker (L1) and an Fc region (F1) and the second polypeptide comprising a second peptide (P2), a linker (L2) and an Fc region (F2). In an aspect of this embodiment, P1 and P2 each independently include a GLP-1, a GLP-1 analogue, a glucagon, a glucacon analogue, a GIP, a GIP analogue, an oxyntomodulin, an oxyntomodulin analogue, an exendin or an exendin analogue. In another aspect of this embodiment, F1 and F2 each independently include an IgG Fc, an IgA Fc, an IgE Fc, an IgGM Fc, or an analogue thereof. In yet another aspect of this embodiment, the C-terminals of peptides P1 and P2 are linked, though the Linkers L1 and L2, to the N-terminals of the Fc regions F1 and F2.

In one embodiment, the disclosed polypeptides form a dimer. If both chains of the dimer are identical, then the present composition may form a homodimer. If each chain of the composition is different, then the present composition may form a heterodimer. The chains may differ in either the peptide (P1/P2) region or in the Fc regions. In one embodiment, the peptides P1 and P2 differ but the Fc regions are identical. In another embodiment, the P1 and P2 differ and the Fc regions differ. Finally, in one embodiment, the P1 and P2 chains may be identical, but the Fc regions of each chain may differ. Furthermore, the Linkers L1 and L2 may be the same or different.

The disclosed fusion proteins include peptides P1 and/or P2. In one embodiment, the present peptides P1 and P2 are each independently selected from a GLP-1, a GLP-1 analogue, a glucagon, a glucacon analogue, a GIP, a GIP analogue, an oxyntomodulin, an oxyntomodulin analogue, an exendin or an exendin analogue. In aspects where the disclosed polypeptides are the same (P1=P2), the polypeptides may have the same amino acid sequence or may have different amino acid sequences.

Analogue as used herein denotes a peptide, polypeptide, or protein sequence which differs from a reference peptide, polypeptide, or protein sequence. Such differences may be the addition, deletion, or substitution of amino acids, phosphorylation, sulfation, acrylation, glycosylation, methylation, farnesylation, acetylation, amidation, and the like, the use of non-natural amino acid structures, or other such modifications as known in the art.

The present composition encompasses amino acid substitutions in proteins and peptides, which do not generally alter the activity of the proteins or peptides (H. Neurath, R. L. Hill, The Proteins, Academic Press, New York, 1979). In one embodiment, these substitutions are “conservative” amino acid substitutions. The most commonly occurring substitutions are Ala/Ser, Val/Ile, Asp/Glu, Thr/Ser, Ala/Gly, Ala/Thr, Ser/Asn, Ala/Val, Ser/Gly, Ala/Pro, Lys/Arg, Asp/Asn, Leu/Ile, Leu/Val, Ala/Glu and Asp/Gly, in both directions.

The term “unnatural amino acids” as used herein refers to amino acids other than the 20 typical amino acids found in the proteins in our human body. Unnatural amino acids are non-proteinogenic amino acids that either occur naturally or are chemically synthesized. They may include but are not limited to aminoisobutyric acid (Aib), β-amino acids (β³ and β²), homo-amino acids, Proline and Pyruvic acid derivatives, 3-substituted Alanine derivatives. Glycine derivatives, Ring-substituted Phenylalanine and Tyrosine derivatives, Linear core amino acids, diamino acids, D-amino acids and N-methyl amino acids.

Further an N-terminal amino acid may be modified by coupling an imidazolic group to the N-terminal amino acid of a polypeptide. Such imidzolic groups can be 4-imidazopropionyl (des-amino-histidyl), 4-amidzoacetyl, 5-imidazo-α, α dimethyl-acetyl. Coupling the imidazolic group to the present fusion peptide or portions thereof may be accomplished by synthetic chemical means. Because many of the various organic groups contemplated herein contain a carboxylic acid, the imidazolic group can be added by solid phase protein synthesis analogous to adding an amino acid to the N-terminus of a polypeptide. Alternatively, an activated ester of the imidazolic group can be added by standard chemical reaction methods. Notation for these imidazolic groups may be denoted by “CA-” appearing prior to the N-termial of a peptide or protein. In one embodiment, the imidazolic group is a 4-imidzoacetyl group.

Amino acid substitutions may be denoted in a variety of ways, but in particular are denoted herein by the format “X^{number}” where X is the substituted amino acid, and the number in superscript is the position of the amino acid to be substituted, or “X_{original} Number X_{substituted},” where “X_{original}” is the original amino acid (in three-letter or one letter format), “Number” is the position of the amino acid to be substituted, and “X_{substituted}” is the substituted amino acid (in three-letter or one letter format). For example, Glu²²-GLP-1(7-37)OH designates a GLP-1 compound in which the glycine normally found at position 22 of GLP-1(7-37)OH has been replaced with glutamic acid; Aib⁸-Glu²²-GLP-1(7-37)OH designates a GLP-1 compound in which alanine normally found at position 8 and glycine normally found at position 22 of GLP-1(7-37)OH have been replaced with Aib and glutamic acid, respectively. These changes may also be denoted as Gly22Glu GLP-1(7-37)OH or G22E GLP-1(7-37)OH.

The term “sequence homology” or “sequence identity” as used herein refers to the percentage of sequence identity between two polypeptide sequences. In order to determine the percentage of identity between two polypeptide sequences, the amino acid sequences of such two sequences are aligned, preferably using the Clustal W algorithm (Thompson, J D, Higgins D G, Gibson T J, 1994, Nucleic Acids Res. 22 (22): 4673-4680), together with BLOSUM 62 scoring matrix (Henikoff and Henikoff, Proc. Natl. Acad. Sci. USA 89: 10915-10919, 1992) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (J. Mol. Biol. 48: 443, 1970), as revised by Smith and Waterman (Adv. Appl. Math. 2: 482, 1981) so that the highest order match is obtained between the two sequences and the number of identical amino acids is determined between the two sequences. Other methods to calculate the percentage identity between two amino acid sequences are generally art recognized and include, for example, those described by Carillo and Lipton (SIAM J. Applied Math. 48: 1073, 1988) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12: 387) BLASTP, BLASTN and FASTA (Altschul et al., J. Molec. Biol., 1990: 215: 403). In one aspect the present modified fusion proteins have at least 70%, at least 75%, at least 80%, at least 85%, at least 87%, at least 90%, at least 91%, at least 92%, at least 93%, at least 94% at least 95%, at least 96%, at least 97%, at least 98%, at least 99% or 100% sequence identity with another sequence, either on a local or a full-length basis.

In one embodiment, GLP-1, glucagon, and oxyntomodulin are obtained from pre-proglucagon. The prepro-glucagon sequence contains within it the peptides for glucagon, glucagon-like peptide 1, glucagon like peptide 2, oxyntomodulin. The pre-proglucagon peptide can be divided into the various mature protein sequences as shown in Table 1. In one embodiment, the pre-proglucagon is SEQ ID NO: 2.

TABLE 1
Pre-ProGlucagon Protein

| Position(s) | Length | Description |
|---|---|---|
| 1-20 | 20 | Signal Peptide |
| 21-89 | 69 | Glicentin |
| 21-50 | 30 | Glicentin-related polypeptide |
| 53-89 | 37 | Oxyntomodulin |
| 53-81 | 29 | Glucagon |
| 84-89 | 6 | Propeptide |
| 92-128 | 37 | Glucagon-like peptide 1 |
| 98-128 | 31 | Glucagon-like peptide 1(7-37) |
| 98-127 | 30 | Glucagon-like peptide 1(7-36) |
| 131-145 | 15 | Propeptide |
| 146-178 | 33 | Glucagon-Like Peptide 2 |

GLP-1 can be found within pre-proglucagon. Notably there is a variation in GLP-1 at the pre-proglucagon position of 115. That is there is an A115V substitution. For instance, GLP-1 can be SEQ ID NO: 3 or SEQ ID NO: 4. Similarly, GLP-1 can be SEQ ID NO: 5, SEQ ID NO: 6, SEQ ID NO: 7 or SEQ ID NO: 8.

The term “GLP-1 analogue” as used herein refers to polypeptides which have sufficient homology to GLP-1, or a fragment of GLP-1, such that the analogue has insulinotropic activity. A number of GLP-1 analogues have been described and disclosed in literature and in patent application and patents, such as patent EP 1641823, the content of which is hereby expressly incorporated by reference in its entirety for all purposes. In one embodiment, a GLP-1 analogue has an amino acid sequence having one, two, three, four, or five amino acids differ from the amino acid in corresponding position of SEQ ID NO: 3, SEQ ID NO: 4, SEQ ID NO: 5, SEQ ID NO: 6, SEQ ID NO: 7 or SEQ ID NO: 8. A GLP-1 analogue sequence can be HXEGTFTSDVSSYLEEQAALEFIAWL VKGGG, wherein X may be aminoisobutyric acid (Aib), Gly, D-Ala, D-Ser, D-Gly, or D-Val (SEQ ID NO: 14); HXEGTFTSDVSSYLEEQAALEFIAWLKNGGG, wherein X is selected from aminoisobutyric acid (Aib), Gly, D-Ala, D-Ser, D-Gly, and D-Val (SEQ ID NO: 15); HXEGTFTSDVSSYLEEQAALEFIAWLVLGGP, wherein X is selected from aminoisobutyric acid (Aib), Gly, D-Ala, D-Ser, D-Gly, and D-Val (SEQ ID NO: 16); HXEGTFTSDVSSYLEEQAALEFIAWLKNGGP, wherein X is selected from Gly, aminoisobutyric acid (Aib), D-Ala, D-Ser, D-Gly, and D-Val (SEQ ID NO: 17); HXEGTFTSDVSSYLEEQAALEFIAWLVKGG, wherein X is selected from Gly, aminoisobutyric acid (Aib), D-Ala, D-Ser, D-Gly, and D-Val (SEQ ID NO: 18); or HXEGTFTSDVSSYLEEQAALEFIAWLKNGG, wherein X is selected from Gly, aminoisobutyric acid (Aib), D-Ala, D-Ser, D-Gly, and D-Val (SEQ ID NO: 19).

In an embodiment, a P1 and/or P2 disclosed herein comprises a GLP-1 peptide or a GLP-1 peptide analogue. In aspects of this embodiment, a P1 and/or P2 disclosed herein comprises SEQ ID NO: 3, SEQ ID NO: 4, SEQ ID NO: 5, SEQ ID NO: 6, SEQ ID NO: 7, SEQ ID NO: 8, SEQ ID NO: 14, SEQ ID NO: 15, SEQ ID NO: 16, SEQ ID NO: 17, SEQ ID NO: 18 or SEQ ID NO: 19. In aspects of this embodiment, a P1 and/or P2 disclosed herein has an amino acid identity of, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 3, SEQ ID NO: 4, SEQ ID NO: 5, SEQ ID NO: 6, SEQ ID NO: 7, SEQ ID NO: 8, SEQ ID NO: 14, SEQ ID NO: 15, SEQ ID NO: 16, SEQ ID NO: 17, SEQ ID NO: 18 or SEQ ID NO: 19. In yet other aspects of this embodiment, a P1 and/or P2 disclosed herein has an amino acid identity in the range of, e.g., about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to SEQ ID NO: 3, SEQ ID NO: 4, SEQ ID NO: 5, SEQ ID NO: 6, SEQ ID NO: 7, SEQ ID NO: 8, SEQ ID NO: 14, SEQ ID NO: 15, SEQ ID NO: 16, SEQ ID NO: 17, SEQ ID NO: 18 or SEQ ID NO: 19.

In other aspects of this embodiment, a P1 and/or P2 disclosed herein has, e.g., at least 1, at least 2, at least 3, at least 4, or at least 5 contiguous amino acid deletions, additions, and/or substitutions relative to SEQ ID NO: 3, SEQ ID NO: 4, SEQ ID NO: 5, SEQ ID NO: 6, SEQ ID NO: 7, SEQ ID NO: 8, SEQ ID NO: 14, SEQ ID NO: 15, SEQ ID NO: 16, SEQ ID NO: 17, SEQ ID NO: 18 or SEQ ID NO: 19; or at most 1, at most 2, at most 3, at most 4, or at most 5 contiguous amino acid deletions, additions, and/or substitutions relative to SEQ ID NO: 3, SEQ ID NO: 4, SEQ ID NO: 5, SEQ ID NO: 6, SEQ ID NO: 7, SEQ ID NO: 8, SEQ ID NO: 14, SEQ ID NO: 15, SEQ ID NO: 16, SEQ ID NO: 17, SEQ ID NO: 18 or SEQ ID NO: 19. In yet other aspects of this embodiment, a P1 and/or P2 disclosed herein has, e.g., at least 1, at least 2, at least 3, at least 4, or at least 5 non-contiguous amino acid deletions, additions, and/or substitutions relative to SEQ ID NO: 3, SEQ ID NO: 4, SEQ ID NO: 5, SEQ ID NO: 6, SEQ ID NO: 7, SEQ ID NO: 8, SEQ ID NO: 14, SEQ ID NO: 15, SEQ ID NO: 16, SEQ ID NO: 17, SEQ ID NO: 18 or SEQ ID NO: 19; or at most 1, at most 2, at most 3, at most 4, or at most 5 non-contiguous amino acid deletions, additions, and/or substitutions relative to SEQ ID NO: 3, SEQ ID NO: 4, SEQ ID NO: 5, SEQ ID NO: 6, SEQ ID NO: 7, SEQ ID NO: 8, SEQ ID NO: 14, SEQ ID NO: 15, SEQ ID NO: 16, SEQ ID NO: 17, SEQ ID NO: 18 or SEQ ID NO: 19.

GIP can be found within the pre-GIP peptide sequence. In aspects of this embodiment, GIP can be SEQ ID NO: 9. The term “GIP analogue” as used herein refers to polypeptides which have sufficient homology to GIP, or a fragment of GIP, such as GIP (1-30) and GIP (7-30), such that the analogue has insulinotropic activity. A significant number of GIP analogues have been described and/or disclosed in PCT publication WO 1998/024464, PCT publication WO 2003/082898, and US Patent Application Publication US 2011/0144007, the content of each of which is hereby expressly incorporated by reference in its entirety for all purposes. In one embodiment, a GIP analogue has one, two, three, four, or five amino acids differ from the amino acid in corresponding position of SEQ ID NO: 9. GIP analogue includes an amino acid sequence YXEGTFISDYSIAMDKIH QQDFVNWLLAQKGKKNDWKHNITQ wherein X is selected from Gly, Val, Lys, and Ser, aminoisobutyric acid (Aib), D-Ala, D-Ser, D-Gly, and D-Val (SEQ ID NO: 20); or YXEGTFISDYSIAMDKIHQQDFVNWLLA QK wherein X is selected from Gly, Val, Lys, and Ser, aminoisobutyric acid (Aib), D-Ala, D-Ser, D-Gly, and D-Val (SEQ ID NO: 21).

In an embodiment, a P1 and/or P2 disclosed herein comprises a GIP peptide or a GIP peptide analogue. In aspects of this embodiment, a P1 and/or P2 disclosed herein comprises SEQ ID NO: 9, SEQ ID NO: 20 or SEQ ID NO: 21. In aspects of this embodiment, a P1 and/or P2 disclosed herein has an amino acid identity of, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 9, SEQ ID NO: 20 or SEQ ID NO: 21. In yet other aspects of this embodiment, a P1 and/or P2 disclosed herein has an amino acid identity in the range of, e.g., about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to SEQ ID NO: 9, SEQ ID NO: 20 or SEQ ID NO: 21.

In other aspects of this embodiment, a P1 and/or P2 disclosed herein has, e.g., at least 1, at least 2, at least 3, at least 4, or at least 5 contiguous amino acid deletions, additions, and/or substitutions relative to SEQ ID NO: 9, SEQ ID NO: 20 or SEQ ID NO: 21; or at most 1, at most 2, at most 3, at most 4, or at most 5 contiguous amino acid deletions, additions, and/or substitutions relative to SEQ ID NO: 9, SEQ ID NO: 20 or SEQ ID NO: 21. In yet other aspects of this embodiment, a P1 and/or P2 disclosed herein has, e.g., at least 1, at least 2, at least 3, at least 4, or at least 5 non-contiguous amino acid deletions, additions, and/or substitutions relative to SEQ ID NO: 9, SEQ ID NO: 20 or SEQ ID NO: 21; or at most 1, at most 2, at most 3, at most 4, or at most 5 non-contiguous amino acid deletions, additions, and/or substitutions relative to SEQ ID NO: 9, SEQ ID NO: 20 or SEQ ID NO: 21.

Exendin as used herein includes polypeptides of the exendin family. Such polypeptides include Exendin-3 and/or Exendin 4. In one embodiment, Exendin-3 corresponds to SEQ ID NO: 10. In one embodiment, Exendin-4 corresponds to SEQ ID NO: 11. The term “exendin analogue” as used herein refers to polypeptides which have sufficient homology to Exendin-3, Exendin-4, or a fragment of Exendin-3 or Exendin-4, such that the analogue has the ability to bind their respective receptors, and/or bind the GLP-1 receptor to inhibit insulin release and maintain blood glucose. A number of exendin analogues have been described and/or disclosed in U.S. Pat. No. 5,424,286, the content of which is hereby expressly incorporated by reference in its entirety for all purposes. In one embodiment, an “exendin analogue” includes a peptide having one, two, three, four or five amino acids which differ from the amino acid in corresponding position of SEQ ID NO: 10, SEQ ID NO: 11, or the peptide with the amino acid sequence SEQ ID NO: 22.

In an embodiment, a P1 and/or P2 disclosed herein comprises an Exendin-3 peptide, an Exendin-4 peptide, an Exendin-3 peptide analogue or an Exendin-4 peptide analogue. In aspects of this embodiment, a P1 and/or P2 disclosed herein comprises SEQ ID NO: 10, SEQ ID NO: 11 or SEQ ID NO: 22. In aspects of this embodiment, a P1 and/or P2 disclosed herein has an amino acid identity of, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 10, SEQ ID NO: 11 or SEQ ID NO: 22. In yet other aspects of this embodiment, a P1 and/or P2 disclosed herein has an amino acid identity in the range of, e.g., about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to SEQ ID NO: 10, SEQ ID NO: 11 or SEQ ID NO: 22.

In other aspects of this embodiment, a P1 and/or P2 disclosed herein has, e.g., at least 1, at least 2, at least 3, at least 4, or at least 5 contiguous amino acid deletions, additions, and/or substitutions relative to SEQ ID NO: 10, SEQ ID NO: 11 or SEQ ID NO: 22; or at most 1, at most 2, at most 3, at most 4, or at most 5 contiguous amino acid deletions, additions, and/or substitutions relative to SEQ ID NO: 10, SEQ ID NO: 11 or SEQ ID NO: 22. In yet other aspects of this embodiment, a P1 and/or P2 disclosed herein has, e.g., at least 1, at least 2, at least 3, at least 4, or at least 5 non-contiguous amino acid deletions, additions, and/or substitutions relative to SEQ ID NO: 10, SEQ ID NO: 11 or SEQ ID NO: 22; or at most 1, at most 2, at most 3, at most 4, or at most 5 non-contiguous amino acid deletions, additions, and/or substitutions relative to SEQ ID NO: 10, SEQ ID NO: 11 or SEQ ID NO: 22.

Glucagon can be found within the pre-proglucagon peptide. In one embodiment, Glucagon corresponds to SEQ ID NO: 12. The term “Glucagon analogue” as used herein refers to polypeptides which have sufficient homology to glucagon or a fragment of glucagon (1-29), such that the analogue has the ability to activate glucagon receptor, counteract the action of insulin and maintain blood glucose levels. A number of glucagon analogues have been described and/or disclosed in U.S. Pat. No. 8,669,228, the content of which is hereby expressly incorporated by reference in its entirety for all purposes. In one embodiment, a Glucagon analogue has one, two, three, four, or five amino acids differ from the amino acid in corresponding position of SEQ ID NO: 12.

In an embodiment, a P1 and/or P2 disclosed herein comprises a Glucagon peptide or a Glucagon peptide analogue. In aspects of this embodiment, a P1 and/or P2 disclosed herein comprises SEQ ID NO: 12. In aspects of this embodiment, a P1 and/or P2 disclosed herein has an amino acid identity of, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 12. In yet other aspects of this embodiment, a P1 and/or P2 disclosed herein has an amino acid identity in the range of, e.g., about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to SEQ ID NO: 12.

In other aspects of this embodiment, a P1 and/or P2 disclosed herein has, e.g., at least 1, at least 2, at least 3, at least 4, or at least 5 contiguous amino acid deletions, additions, and/or substitutions relative to SEQ ID NO: 12; or at most 1, at most 2, at most 3, at most 4, or at most 5 contiguous amino acid deletions, additions, and/or substitutions relative to SEQ ID NO: 12. In yet other aspects of this embodiment, a P1 and/or P2 disclosed herein has, e.g., at least 1, at least 2, at least 3, at least 4, or at least 5 non-contiguous amino acid deletions, additions, and/or substitutions relative to SEQ ID NO: 12; or at most 1, at most 2, at most 3, at most 4, or at most 5 non-contiguous amino acid deletions, additions, and/or substitutions relative to SEQ ID NO: 12.

In one embodiment, Oxyntomodulin can be found within the pre-proglucagon peptide. In one embodiment, Oxyntomodulin corresponds to SEQ ID NO: 13. The term “oxyntomodulin analogue” as used herein refers to polypeptides which have sufficient homology to Oxynotomodulin or a fragment of Oxyntomodulin such that the analogue has the ability to activate both GIP-1 and glucagon receptors. A number of oxyntomodulin analogues have been described and/or disclosed in PCT publication WO 2012/173422, the content of which is hereby expressly incorporated by reference in its entirety for all purposes. In one embodiment, an Oxyntomodulin analogue has one, two, three, four, or five amino acids differ from the amino acid in corresponding position of SEQ ID NO: 13. Further, Oxyntomodulin is a 37 amino acid peptide that contains the 29 amino acid sequence of glucagon followed by an 8 amino acid carboxy terminal extension of SEQ ID NO: 41 (KRNRNNIA). Thus, it is possible that analogues of glucagon may be further modified by adding the 8 amino acid carboxy terminal extension of oxyntomodulin.

In one embodiment, the oxyntomodulin analog can be one of the following sequences HXQGTFTSDYSKYLDEKRAKEFVQWLMNT, wherein X is an amino isobutyric acid (SEQ ID NO: 23); HXQGTFTSDYSKYLDEKRAKEFVQWLMNT wherein X is an amino isobutyric acid (SEQ ID NO: 24); HXQGTFTSDYSKYLDEQAAKEFICWLMNT, wherein X is an amino isobutyric acid (SEQ ID NO: 25); HXQGTFTSDYSKYLDEKRAKEFVQWLMNT, wherein X is an amino isobutyric acid (SEQ ID NO: 26); XXQGTFTSDYSKYLDEKRAKEFVQWLMNT, wherein X and X in the first position represents 4-imidazoacetyl (i.e., CA) and X in the second position is an amino isobutyric acid. (SEQ ID NO: 27); HXQGTFTSDYAKYLDEKRAKEFVQWLMNT, wherein X is an amino isobutyric acid (SEQ ID NO: 28); HSQGTFTSDYSKYLDSRRAQDFVQWLMNTKRNRNNIA, wherein the N-terminal H may or may not be the D enantiomer of Histidine (SEQ ID NO: 29); YXQGTFTSDYSKYLDEKRAKEFVQWLMNT, wherein X is an amino isobutyric acid (SEQ ID NO: 30) wherein amino acids in bold and underlined represent ring formation, and wherein X is an amino isobutyric acid.

In an embodiment, a P1 and/or P2 disclosed herein comprises an oxyntomodulin peptide or an oxyntomodulin peptide analogue. In aspects of this embodiment, a P1 and/or P2 disclosed herein comprises SEQ ID NO: 13, SEQ ID NO: 23, SEQ ID NO: 24, SEQ ID NO: 25, SEQ ID NO: 26, SEQ ID NO: 27, SEQ ID NO: 28, SEQ ID NO: 29 or SEQ ID NO: 30. In aspects of this embodiment, a P1 and/or P2 disclosed herein has an amino acid identity of, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 13, SEQ ID NO: 23, SEQ ID NO: 24, SEQ ID NO: 25, SEQ ID NO: 26, SEQ ID NO: 27, SEQ ID NO: 28, SEQ ID NO: 29 or SEQ ID NO: 30. In yet other aspects of this embodiment, a P1 and/or P2 disclosed herein has an amino acid identity in the range of, e.g., about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to SEQ ID NO: 13, SEQ ID NO: 23, SEQ ID NO: 24, SEQ ID NO: 25, SEQ ID NO: 26, SEQ ID NO: 27, SEQ ID NO: 28, SEQ ID NO: 29 or SEQ ID NO: 30.

In other aspects of this embodiment, a P1 and/or P2 disclosed herein has, e.g., at least 1, at least 2, at least 3, at least 4, or at least 5 contiguous amino acid deletions, additions, and/or substitutions relative to SEQ ID NO: 13, SEQ ID NO: 23, SEQ ID NO: 24, SEQ ID NO: 25, SEQ ID NO: 26, SEQ ID NO: 27, SEQ ID NO: 28, SEQ ID NO: 29 or SEQ ID NO: 30; or at most 1, at most 2, at most 3, at most 4, or at most 5 contiguous amino acid deletions, additions, and/or substitutions relative to SEQ ID NO: 13, SEQ ID NO: 23, SEQ ID NO: 24, SEQ ID NO: 25, SEQ ID NO: 26, SEQ ID NO: 27, SEQ ID NO: 28, SEQ ID NO: 29 or SEQ ID NO: 30. In yet other aspects of this embodiment, a P1 and/or P2 disclosed herein has, e.g., at least 1, at least 2, at least 3, at least 4, or at least 5 non-contiguous amino acid deletions, additions, and/or substitutions relative to SEQ ID NO: 13, SEQ ID NO: 23, SEQ ID NO: 24, SEQ ID NO: 25, SEQ ID NO: 26, SEQ ID NO: 27, SEQ ID NO: 28, SEQ ID NO: 29 or SEQ ID NO: 30; or at most 1, at most 2, at most 3, at most 4, or at most 5 non-contiguous amino acid deletions, additions, and/or substitutions relative to SEQ ID NO: 13, SEQ ID NO: 23, SEQ ID NO: 24, SEQ ID NO: 25, SEQ ID NO: 26, SEQ ID NO: 27, SEQ ID NO: 28, SEQ ID NO: 29 or SEQ ID NO: 30.

In one embodiment, P1 and/or P2 may have an N-terminal amino acid of His or dHis (the D enantiomer of histidine) added thereon or substituted for the naturally occurring N-terminal amino acid, and the second amino acid from the N-terminal of P1 or P2 may be Gly, aminoisobutyric acid (Aib), D-Ala, D-Ser, D-Gly, and D-Val. In one particular aspect of this embodiment, the P1 is a GIP-1 analogue. In a further aspect of this embodiment, the P1 is a GIP, a GIP analogue, a Glucagon, or a Glucagon analogue.

The present fusion proteins may act as a dual receptor agonist. The term “dual receptor agonist” or “dual receptor co-agonist” as used herein refers to a peptide or a fusion protein fusion protein which is capable of activating two receptors selected from GLP-1 receptor, GIP receptor and Glucagon receptor.

P1 and/or P2 may act as a unimolecular dual receptor agonist alone, or when fused to the Fc. The term “unimolecular dual receptor co-agonist” as used herein refers to a single polypeptide which possesses agonist activities to at least two different receptors. For example, unimolecular Glucagon/GLP-1 receptor co-agonists have been disclosed in U.S. Pat. No. 8,454,971, the content of which is hereby expressly incorporated by reference in its entirety for all purposes, and described by Finan et al (Finan et al: “Unimolecular dual incretins maximize metabolic benefits in rodents, monkeys, and humans.” Sci Transl Med. 2013 Oct. 30; 5(209):209). In this manner, the present fusion proteins may be dual receptor agonists, or even triple receptor agonists. For instance, if the P1 was a unimolecular dual receptor agonist, and the P2 was a single receptor agonist, the fusion protein may demonstrate agonist activity against three receptors. In one aspect of this embodiment, the fusion protein may be a dual agonist and have agonist activity against any two of the GLP-1 receptor, the GIP receptor, and the Glucagon receptor. In another aspect of this embodiment, the fusion protein may be a triple agonist and have agonist activity against the GLP-1 receptor, the GIP receptor, and the Glucagon receptor.

It is to be noted that a single P1 or P2 may demonstrate agonist activity against a first receptor, and yet have antagonistic or inhibitory activity against a second receptor. Furthermore, in one embodiment, P1 may have an agonist activity against one receptor while P2 has an antagonist activity against a second receptor.

Various combinations of P1 and P2 are contemplated. In one embodiment, P1 and P2 sequences are chosen independently from any one of SEQ ID NOs: 3-30, an amino acid sequence having one, two, three, four or five amino acid additions, deletions or substitutions when compared to SEQ ID NOs: 3-30, or an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to one or more of SEQ ID NOs: 3-30.

In one embodiment, at least one of the peptides P1 or P2 in the fusion protein is a GLP-1 analogue, A GLP-1 analogue having an amino acid sequence selected from SEQ ID NOs: 14-19, an amino acid sequence having one, two, three, four or five amino acid additions, deletions or substitutions when compared to SEQ ID NOs: 14-19, or an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to one or more of SEQ ID NOs: 14-19.

In a further embodiment, the P1 in the fusion protein is a GLP-1 analogue, and the P2 in the fusion protein is a GIP analogue. In an aspect of this embodiment, the GLP-1 analogue may have an amino acid sequence selected from SEQ IDs NO: 14-19 and the GIP analogue may contains an amino acid sequence selected from those described and disclosed in PCT publication WO 1998/024464, PCT publication WO 2003/082898, and US Patent Application Publication US 2011/0144007, the content of which is hereby expressly incorporated by reference in its entirety for all purposes. In yet another aspect of this embodiment, the P1 peptide in the fusion protein is a GLP-1 analogue, and the P2 peptide in the fusion protein is a GIP analogue, wherein the GLP-1 analogue contains an amino acid sequence selected from SEQ IDs NO: 14-19; and the GIP analogue contains an amino acid sequence selected from SEQ ID NO: 20-21.

The present compositions include two “Fc fragments” or “Fc regions,” F1 and F2. F1 and F2 may have the same amino acid sequence or different amino acid sequences. In one embodiment, F1 and F2 are capable of dimerization. The term “Fc fragment” or “immunoglobulin Fc region” as used herein, refers to a protein that contains at least the heavy-chain constant region 2 (CH2) and the heavy-chain constant region 3 (CH3) of an immunoglobulin. In one embodiment, the Fc region excludes the variable regions of the heavy and light chains, the heavy-chain constant region 1 (CH1) and the light-chain constant region 1 (CL1) of the immunoglobulin. The Fc region may further include a hinge region at the heavy-chain constant region. Also, the immunoglobulin Fc region disclosed herein may contain a part or all of the Fc region including the heavy-chain constant region 1 (CH1) and/or the light-chain constant region 1 (CL1), except for the variable regions of the heavy and light chains, as long as it has a physiological function substantially similar to or better than the native protein. Also, the immunoglobulin Fc region may be a fragment having a deletion in a relatively long portion of the amino acid sequence of CH2 and/or CH3. That is, the immunoglobulin Fc region disclosed herein may comprise 1) a CH1 domain, a CH2 domain, a CH3 domain and a CH4 domain, 2) a CH1 domain and a CH2 domain, 3) a CH1 domain and a CH3 domain, 4) a CH2 domain and a CH3 domain, 5) a combination of one or more domains and an immunoglobulin hinge region (or a portion of the hinge region), and 6) a dimer of each domain of the heavy-chain constant regions and the light-chain constant region.

The immunoglobulin Fc region disclosed herein includes a native amino acid sequence, or a sequence analogue thereof. An amino acid sequence analogue is a sequence that is different from the native amino acid sequence due to a deletion, an insertion, a non-conservative or conservative substitution or combinations thereof of one or more amino acid residues.

Also, other various analogues are possible, including one in which a region capable of forming a disulfide bond is deleted, or certain amino acid residues are eliminated at the N-terminal end of a native Fc form or a methionine residue is added thereto. Further, to remove effector functions, a deletion may occur in a complement-binding site, such as a C1q-binding site and an ADCC (antibody dependent cell mediated cytotoxicity) site. Techniques of preparing such sequence analogues of the immunoglobulin Fc region are disclosed in WO 1997/034631 and WO 1996/032478.

The aforementioned Fc analogues are analogues that have a biological activity identical to the Fc region disclosed herein or improved structural stability, for example, against heat, pH, or the like.

In addition, these Fc regions may be obtained from native forms isolated from humans and other animals including cows, goats, pigs, mice, rabbits, hamsters, rats and guinea pigs, or may be recombinants or analogues thereof, obtained from transformed animal cells or microorganisms. Herein, they may be obtained from a native immunoglobulin by isolating whole immunoglobulins from human or animal organisms and treating them with a proteolytic enzyme. Papain digests the native immunoglobulin into Fab and Fc regions, and pepsin treatment results in the production of pF'c and F(ab)2 fragments. These fragments may be subjected, for example, to size exclusion chromatography to isolate Fc or pF'c. Preferably, a human-derived Fc region is a recombinant immunoglobulin Fc region that is obtained from a microorganism.

In one embodiment, the Fc region, if desired, may be modified by phosphorylation, sulfation, acrylation, glycosylation, methylation, farnesylation, acetylation, amidation, and the like. In one embodiment, the immunoglobulin Fc region disclosed herein may be in the form of having native sugar chains, increased sugar chains compared to a native form or decreased sugar chains compared to the native form, or may be in a deglycosylated form. The increase, decrease or removal of the immunoglobulin Fc sugar chains may be achieved by methods common in the art, such as a chemical method, an enzymatic method and a genetic engineering method using a microorganism. The removal of sugar chains from an Fc region results in a sharp decrease in binding affinity to the C1q part of the first complement component C1 and a decrease or loss in antibody-dependent cell-mediated cytotoxicity or complement-dependent cytotoxicity, thereby not inducing unnecessary immune responses in-vivo. In this regard, an immunoglobulin Fc region in a deglycosylated or aglycosylated form may be more suitable as a drug carrier.

As used herein, the term “deglycosylation” refers to enzymatically removing sugar moieties from an Fc region, and the term “aglycosylation” means that an Fc region is produced in an unglycosylated form by a prokaryote, preferably E. coli.

In one embodiment, the immunoglobulin Fc region may be an Fc region that is derived from IgG, IgA, IgD, IgE and IgM, or that is made by combinations thereof or hybrids thereof. Preferably, it is derived from IgG or IgM, which are among the most abundant proteins in human blood, and most preferably from IgG, which is known to enhance the half-lives of ligand-binding proteins.

On the other hand, the term “Fc combination”, as used herein, means that polypeptides encoding single-chain immunoglobulin Fc regions of the same origin are linked to a single-chain polypeptide of a different origin to form a dimer or multimer. That is, a dimer or multimer may be formed from two or more fragments selected from the group consisting of IgG Fc, IgA Fc, IgM Fc, IgD Fc, and IgE Fc fragments.

The term “IgG4 Fc analogues” as used herein refers to polypeptides which have sufficient homology to IgG4 (218-437) (see e.g., the IgG4 Fc sequence SEQ ID NO: 31, amino acids 101-317).

Meanwhile, the Fc fragment or the Fc region of the dummy antibody may be modified to increase its affinity with neonatal Fc receptor (FcRN) and further extend its half-life in vivo. As mentioned above, Fc is often used as carrier to extend the in vivo half-lives of therapeutic peptides such as GLP-1 analogues. Fc fusion proteins has in vivo half-lives ranged from a few days to over a week. Meanwhile, full antibody molecule may also be used as carrier for therapeutic peptides. Antibody molecules can have in vivo half-lives over 3 weeks. In addition, modifications can be made to the Fc domain to further extend half-life in vivo of an antibody molecule or an Fc fusion protein. For example, introduction of mutations T250Q/M428L to an IgG1 molecule led to a significant increase in the serum half-life of the IgG1 molecule, also as disclosed in U.S. Pat. No. 7,217,798, the content of which is hereby expressly incorporated by reference in its entirety for all purposes. Other mutations have also been made to increase the binding affinity of Fc to FcRN and extend the serum half-life, such as disclosed in U.S. Pat. No. 8,394,925, the content of which is hereby expressly incorporated by reference in its entirety for all purposes.

Another modification, referred as “knob-and-hole” may also be made to the Fc region when necessary in order to form heterogeneous dimers. Antibody molecules have heavy chains and light chains. It's convenient to fuse two different peptides to an antibody molecule. Fc fusion proteins are often homo-dimers. However, hetero-dimers can also be constructed. For example, U.S. Pat. No. 7,642,228, the content of which is hereby expressly incorporated by reference in its entirety for all purposes, disclosed that heterodimers of antibodies and Fc-fusion proteins can be constructed using the Knob-and-Hole concept. Many of the variants have been described. For examples, the Fc polypeptide comprising the cavity (aka F2) comprises two or more amino acid replacements selected from the group consisting of T366S, L368A, or Y407V, amino acid numbering according to the EU numbering scheme of Kabat; and the Fc polypeptide comprising the protuberance (aka F1) comprises replacement of threonine at position 366 with tryptophan or tyrosine, amino acid numbering according to the EU numbering scheme of Kabat.

For example, the numbering corresponds as shown in Table 2.

TABLE 2
Comparison of Kabat/EU Amino Acid Position to Reference Sequences

| Reference Position | SEQ ID NO: | T→Q or E | T→S, W or Y | L→A | Y→V | M→L or F |
|---|---|---|---|---|---|---|
| Kabat/EU | universal | 250 | 366 | 368 | 407 | 428 |
| IgG4 Fc | 31 | 130 | 246 | 248 | 287 | 308 |
| IgG2 FC | 32 | 129 | 245 | 247 | 286 | 307 |
| IgG1 Fc | 33 | 133 | 249 | 251 | 290 | 311 |

Additional approaches for making heterogeneous dimer can also be made for IgG Fc. For example, introduction of dual point mutations F188L and R192K in one of the IgG4 Fc polypeptide as shown in SEQ ID NO:35 allows it to form a heterodimer with another IgG4 Fc polypeptide without these two mutations.

In one embodiment, the IgG4 Fc includes the amino acid sequence SEQ ID NO: 31, or a fragment thereof. In one embodiment, the IgG4 Fc includes one or more of the following fragments of SEQ ID NO: 31 as shown in Table 3.

TABLE 3
IgG4 Regions

| Amino Acids | Length | Region |
|---|---|---|
| 1-98 | 98 | CH1 |
| 99-110 | 12 | Hinge |
| 111-220 | 110 | CH2 |
| 221-327 | 107 | CH3 |

In one aspect of the present Fc, the Fc includes: 1) an IgG4 CH1 domain, an IgG4 CH2 domain, and an IgG4 CH3 domain, 2) an IgG4 CH1 domain and a CH2 domain, 3) an IgG4 CH1 domain and a CH3 domain, 4) an IgG4 CH2 domain and a CH3 domain, 5) a combination of one or more domains and an immunoglobulin hinge region (or a portion of the hinge region), or 6) a combination of one or more an IgG4 CH domains and CH domains or hinge regions from other immunoglobulin subtypes.

In exemplary but not limiting aspects of this embodiment, the IgG4 Fc may include one or more of the following sequences: the IgG4 CH1 of SEQ ID NO: 42, the IgG4 CH2 of SEQ ID NO: 43, the IgG4 CH3 of SEQ ID NO: 44, the IgG4 CH1 and CH2 (no hinge) of SEQ ID NO: 45, the IgG4 CH1 and CH3 (no hinge) of SEQ ID NO: 46, and/or the IgG4 CH2 and CH3 (no hinge) of SEQ ID NO: 47.

In an embodiment, a F1 and/or F2 disclosed herein comprises an IgG4 Fc or an IgG4 Fc fragment. In aspects of this embodiment, a F1 and/or F2 disclosed herein comprises SEQ ID NO: 31, SEQ ID NO: 42, SEQ ID NO: 43, SEQ ID NO: 44, SEQ ID NO: 45, SEQ ID NO: 46 or SEQ ID NO: 47. In aspects of this embodiment, a F1 and/or F2 disclosed herein has an amino acid identity of, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 31, SEQ ID NO: 42, SEQ ID NO: 43, SEQ ID NO: 44, SEQ ID NO: 45, SEQ ID NO: 46 or SEQ ID NO: 47. In yet other aspects of this embodiment, a F1 and/or F2 disclosed herein has an amino acid identity in the range of, e.g., about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to SEQ ID NO: 31, SEQ ID NO: 42, SEQ ID NO: 43, SEQ ID NO: 44, SEQ ID NO: 45, SEQ ID NO: 46 or SEQ ID NO: 47.

In other aspects of this embodiment, a F1 and/or F2 disclosed herein has, e.g., at least 1, at least 2, at least 3, at least 4, at least 5, at least 6, at least 7, at least 8, at least 9, or at least 10 contiguous amino acid deletions, additions, and/or substitutions relative SEQ ID NO: 31, SEQ ID NO: 42, SEQ ID NO: 43, SEQ ID NO: 44, SEQ ID NO: 45, SEQ ID NO: 46 or SEQ ID NO: 47; or at most 1, at most 2, at most 3, at most 4, at most 5, at most 6, at most 7, at most 8, at most 9, or at most 10 contiguous amino acid deletions, additions, and/or substitutions relative to SEQ ID NO: 31, SEQ ID NO: 42, SEQ ID NO: 43, SEQ ID NO: 44, SEQ ID NO: 45, SEQ ID NO: 46 or SEQ ID NO: 47. In yet other aspects of this embodiment, a F1 and/or F2 disclosed herein has, e.g., at least 1, at least 2, at least 3, at least 4, at least 5, at least 6, at least 7, at least 8, at least 9, or at least 10 non-contiguous amino acid deletions, additions, and/or substitutions relative to SEQ ID NO: 31, SEQ ID NO: 42, SEQ ID NO: 43, SEQ ID NO: 44, SEQ ID NO: 45, SEQ ID NO: 46 or SEQ ID NO: 47; or at most 1, at most 2, at most 3, at most 4, at most 5, at most 6, at most 7, at most 8, at most 9, or at most 10 non-contiguous amino acid deletions, additions, and/or substitutions relative to SEQ ID NO: 31, SEQ ID NO: 42, SEQ ID NO: 43, SEQ ID NO: 44, SEQ ID NO: 45, SEQ ID NO: 46 or SEQ ID NO: 47.

In one embodiment, the IgG1 Fc includes the amino acid sequence of SEQ ID NO: 32 or a fragment thereof. In one embodiment, the IgG4 Fc includes one or more of the following fragments of SEQ ID NO: 32 as shown in Table 4.

TABLE 4
IgG1 Regions

| Amino Acids | Length | Region |
|---|---|---|
| 1-98 | 98 | CH1 |
| 99-110 | 12 | Hinge |
| 111-223 | 113 | CH2 |
| 224-330 | 107 | CH3 |

In one aspect of the present Fc, the Fc includes: 1) an IgG1 CH1 domain, an IgG1 CH2 domain, and an IgG1 CH3 domain, 2) an IgG1 CH1 domain and a CH2 domain, 3) an IgG1 CH1 domain and a CH3 domain, 4) an IgG1 CH2 domain and a CH3 domain, 5) a combination of one or more domains and an immunoglobulin hinge region (or a portion of the hinge region), or 6) a combination of one or more an IgG1 CH domains and CH domains or hinge regions from other immunoglobulin subtypes.

In exemplary but not limiting aspects of this embodiment, the IgG1 Fc may include one or more of the following sequences: the IgG1: CH1 of SEQ ID NO: 48, the IgG1: CH2 of SEQ ID NO: 49, the IgG1: CH3 of SEQ ID NO: 50, the IgG1: CH1 and CH2 (no hinge) of SEQ ID NO: 51, the IgG1: CH1 and CH3 (no hinge) of SEQ ID NO: 52, and/or the IgG1: CH2 and CH3 (no hinge) of SEQ ID NO: 53.

In an embodiment, a F1 and/or F2 disclosed herein comprises an IgG1 Fc or an IgG1 Fc fragment. In aspects of this embodiment, a F1 and/or F2 disclosed herein comprises SEQ ID NO: 32, SEQ ID NO: 48, SEQ ID NO: 49, SEQ ID NO: 50, SEQ ID NO: 51, SEQ ID NO: 52 or SEQ ID NO: 53. In aspects of this embodiment, a F1 and/or F2 disclosed herein has an amino acid identity of, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 32, SEQ ID NO: 48, SEQ ID NO: 49, SEQ ID NO: 50, SEQ ID NO: 51, SEQ ID NO: 52 or SEQ ID NO: 53. In yet other aspects of this embodiment, a F1 and/or F2 disclosed herein has an amino acid identity in the range of, e.g., about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to SEQ ID NO: 32, SEQ ID NO: 48, SEQ ID NO: 49, SEQ ID NO: 50, SEQ ID NO: 51, SEQ ID NO: 52 or SEQ ID NO: 53.

In other aspects of this embodiment, a F1 and/or F2 disclosed herein has, e.g., at least 1, at least 2, at least 3, at least 4, at least 5, at least 6, at least 7, at least 8, at least 9, or at least 10 contiguous amino acid deletions, additions, and/or substitutions relative SEQ ID NO: 32, SEQ ID NO: 48, SEQ ID NO: 49, SEQ ID NO: 50, SEQ ID NO: 51, SEQ ID NO: 52 or SEQ ID NO: 53; or at most 1, at most 2, at most 3, at most 4, at most 5, at most 6, at most 7, at most 8, at most 9, or at most 10 contiguous amino acid deletions, additions, and/or substitutions relative SEQ ID NO: 32, SEQ ID NO: 48, SEQ ID NO: 49, SEQ ID NO: 50, SEQ ID NO: 51, SEQ ID NO: 52 or SEQ ID NO: 53. In yet other aspects of this embodiment, a F1 and/or F2 disclosed herein has, e.g., at least 1, at least 2, at least 3, at least 4, at least 5, at least 6, at least 7, at least 8, at least 9, or at least 10 non-contiguous amino acid deletions, additions, and/or substitutions relative to SEQ ID NO: 32, SEQ ID NO: 48, SEQ ID NO: 49, SEQ ID NO: 50, SEQ ID NO: 51, SEQ ID NO: 52 or SEQ ID NO: 53; or at most 1, at most 2, at most 3, at most 4, at most 5, at most 6, at most 7, at most 8, at most 9, or at most 10 non-contiguous amino acid deletions, additions, and/or substitutions relative to SEQ ID NO: 32, SEQ ID NO: 48, SEQ ID NO: 49, SEQ ID NO: 50, SEQ ID NO: 51, SEQ ID NO: 52 or SEQ ID NO: 53.

In one embodiment, the IgG2 Fc includes the amino acid sequence of SEQ ID NO: 33 or a fragment thereof. In one embodiment, the IgG2 Fc includes one or more of the following fragments of SEQ ID NO: 33 as shown in Table 5.

TABLE 5
IgG2 Regions

| Amino Acids | Length | Region |
|---|---|---|
| 1-98 | 98 | CH1 |
| 99-110 | 12 | Hinge |
| 111-219 | 109 | CH2 |
| 220-326 | 107 | CH3 |

In one aspect of the present Fc, the Fc includes: 1) an IgG2 CH1 domain, an IgG2 CH2 domain, and an IgG2 CH3 domain, 2) an IgG2 CH1 domain and a CH2 domain, 3) an IgG2 CH1 domain and a CH3 domain, 4) an IgG2 CH2 domain and a CH3 domain, 5) a combination of one or more domains and an immunoglobulin hinge region (or a portion of the hinge region), or 6) a combination of one or more an IgG2 CH domains and CH domains or hinge regions from other immunoglobulin subtypes.

In exemplary but not limiting aspects of this embodiment, the IgG2 Fc may include one or more of the following sequences: the IgG2: CH1 of SEQ ID NO: 54, the IgG2: CH2 of SEQ ID NO: 55, the IgG2: CH3 of SEQ ID NO: 56, the IgG2: CH1 and CH2 (no hinge) of SEQ ID NO: 57, the IgG2: CH1 and CH3 (no hinge) of SEQ ID NO: 58, and/or the IgG2: CH2 and CH3 (no hinge) of SEQ ID NO: 59.

In an embodiment, a F1 and/or F2 disclosed herein comprises an IgG2 Fc or an IgG2 Fc fragment. In aspects of this embodiment, a F1 and/or F2 disclosed herein comprises SEQ ID NO: 33, SEQ ID NO: 54, SEQ ID NO: 55, SEQ ID NO: 56, SEQ ID NO: 57, SEQ ID NO: 58 or SEQ ID NO: 59. In aspects of this embodiment, a F1 and/or F2 disclosed herein has an amino acid identity of, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 33, SEQ ID NO: 54, SEQ ID NO: 55, SEQ ID NO: 56, SEQ ID NO: 57, SEQ ID NO: 58 or SEQ ID NO: 59. In yet other aspects of this embodiment, a F1 and/or F2 disclosed herein has an amino acid identity in the range of, e.g., about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to SEQ ID NO: 33, SEQ ID NO: 54, SEQ ID NO: 55, SEQ ID NO: 56, SEQ ID NO: 57, SEQ ID NO: 58 or SEQ ID NO: 59.

In other aspects of this embodiment, a F1 and/or F2 disclosed herein has, e.g., at least 1, at least 2, at least 3, at least 4, at least 5, at least 6, at least 7, at least 8, at least 9, or at least 10 contiguous amino acid deletions, additions, and/or substitutions relative SEQ ID NO: 33, SEQ ID NO: 54, SEQ ID NO: 55, SEQ ID NO: 56, SEQ ID NO: 57, SEQ ID NO: 58 or SEQ ID NO: 59; or at most 1, at most 2, at most 3, at most 4, at most 5, at most 6, at most 7, at most 8, at most 9, or at most 10 contiguous amino acid deletions, additions, and/or substitutions relative SEQ ID NO: 33, SEQ ID NO: 54, SEQ ID NO: 55, SEQ ID NO: 56, SEQ ID NO: 57, SEQ ID NO: 58 or SEQ ID NO: 59. In yet other aspects of this embodiment, a F1 and/or F2 disclosed herein has, e.g., at least 1, at least 2, at least 3, at least 4, at least 5, at least 6, at least 7, at least 8, at least 9, or at least 10 non-contiguous amino acid deletions, additions, and/or substitutions relative to SEQ ID NO: 33, SEQ ID NO: 54, SEQ ID NO: 55, SEQ ID NO: 56, SEQ ID NO: 57, SEQ ID NO: 58 or SEQ ID NO: 59; or at most 1, at most 2, at most 3, at most 4, at most 5, at most 6, at most 7, at most 8, at most 9, or at most 10 non-contiguous amino acid deletions, additions, and/or substitutions relative to SEQ ID NO: 33, SEQ ID NO: 54, SEQ ID NO: 55, SEQ ID NO: 56, SEQ ID NO: 57, SEQ ID NO: 58 or SEQ ID NO: 59.

In one embodiment, the IgM Fc includes the amino acid sequence SEQ ID NO: 34 or a fragment thereof. In one embodiment, the IgM Fc includes one or more of the following fragments of SEQ ID NO: 34 as shown in Table 6.

TABLE 6
IgM Regions

| Amino Acids | Length | Region |
|---|---|---|
| 1-105 | 105 | CH1 |
| 106-217 | 112 | CH2 |
| 218-323 | 106 | CH3 |
| 324-452 | 129 | CH4 |

In one aspect of the present Fc, the Fc includes: 1) an IgM CH1 domain, an IgM CH2 domain, and an IgM CH3 domain, 2) an IgM CH1 domain and a CH2 domain, 3) an IgM CH1 domain and a CH3 domain, 4) an IgM CH2 domain and a CH3 domain, 5) a combination of one or more domains and an immunoglobulin hinge region (or a portion of the hinge region), or 6) a combination of one or more an IgM CH domains and CH domains or hinge regions from other immunoglobulin subtypes.

In exemplary but not limiting aspects of this embodiment, the IgM Fc may include one or more of the following sequences: the IgM: CH1 of SEQ ID NO: 60, the IgM: CH2 of SEQ ID NO: 61, the IgM: CH3 of SEQ ID NO: 62, the IgM: CH4 of SEQ ID NO: 63, the IgM: CH1 and CH2 of SEQ ID NO: 64, the IgM: CH1 and CH3 of SEQ ID NO: 65, the IgM CH1 and CH4 of SEQ ID NO: 66, the IgM CH1-CH3-CH4 of SEQ ID NO: 67, the IgM CH1-CH2-CH4 of SEQ ID NO: 68, the IgM CH2-CH3 of SEQ ID NO: 69, the IgM CH2-CH4 of SEQ ID NO: 70, the IgM CH2-CH3-CH4 of SEQ ID NO: 71 and/or the IgM CH3-CH4 of SEQ ID NO: 72.

In an embodiment, a F1 and/or F2 disclosed herein comprises an IgM Fc or an IgM Fc fragment. In aspects of this embodiment, a F1 and/or F2 disclosed herein comprises SEQ ID NO: 34, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71 or SEQ ID NO: 72. In aspects of this embodiment, a F1 and/or F2 disclosed herein has an amino acid identity of, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 34, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71 or SEQ ID NO: 72. In yet other aspects of this embodiment, a F1 and/or F2 disclosed herein has an amino acid identity in the range of, e.g., about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to SEQ ID NO: 34, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71 or SEQ ID NO: 72.

In other aspects of this embodiment, a F1 and/or F2 disclosed herein has, e.g., at least 1, at least 2, at least 3, at least 4, at least 5, at least 6, at least 7, at least 8, at least 9, or at least 10 contiguous amino acid deletions, additions, and/or substitutions relative SEQ ID NO: 34, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71 or SEQ ID NO: 72; or at most 1, at most 2, at most 3, at most 4, at most 5, at most 6, at most 7, at most 8, at most 9, or at most 10 contiguous amino acid deletions, additions, and/or substitutions relative SEQ ID NO: 34, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71 or SEQ ID NO: 72. In yet other aspects of this embodiment, a F1 and/or F2 disclosed herein has, e.g., at least 1, at least 2, at least 3, at least 4, at least 5, at least 6, at least 7, at least 8, at least 9, or at least 10 non-contiguous amino acid deletions, additions, and/or substitutions relative to SEQ ID NO: 34, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71 or SEQ ID NO: 72; at most 1, at most 2, at most 3, at most 4, at most 5, at most 6, at most 7, at most 8, at most 9, or at most 10 non-contiguous amino acid deletions, additions, and/or substitutions relative to SEQ ID NO: 34, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71 or SEQ ID NO: 72.

In one embodiment, the composition includes a modified Fc having an amino acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% sequence identity to the full-length sequence of SEQ ID NOs: 31-34 or 42-72. In another embodiment, the composition includes a modified Fc having an amino acid sequence of SEQ ID NOs: 31-34 or 42-72 with up to 70, up to 60, up to 50, up to 45, up to 40, up to 35, up to 30, up to 25, up to 20, up to 15, up to 14, up to 13, up to 12, up to 11, up to 10, up to 9, up to 8, up to 7, up to 7, up to 6, up to 5, up to 4, up to 3, up to 2, or up to 1 amino acid amino acid additions, deletions, or substitutions when compared to the full length of SEQ ID NOs: 31-34 or 42-72.

In one embodiment, the Fc region in the Fc fusion protein contains the amino acid sequence AES KYGPPCPPCPAPXXXGGPSVFLFPPKPKDXLXIXRXPEVTCVVVDVSQEDPEVQFNWYVDGVEVHNAKT KPREEQFXSTYRVVSVLTVLHQDWLNGKEYKCKVSNKGLPSSIEKTISKAKGQPREPQVYTLPPSQEEMT KNQVSLTCLVKGFYPSDIAVEWESNGQPENNYKTTPPVLDSDGSFXLYSXLTVDKSRWQEGNVFSCSVX HEALHXHYTQKSLSLSLGX, wherein X at position 16 is Pro or Glu; X at position 17 is Phe, Val, or Ala; X at position 18 is Leu, Glu, or Ala; X at position 33 is Thr, Glu, or Gln; X at position 35 is Met or Trp; X at position 37 is Ser or Thr; X at position 39 is Thr or Glu; X at position 80 is Asn or Ala; X at position 188 is Phe or Leu; X at position 192 is Lys or Arg; X at position 211 is Leu, Met, Ala or Phe; X at position 217 is Asn or Ser; and X at position 230 is Lys or is absent (SEQ ID NO: 35).

In an embodiment, a F1 and/or F2 disclosed herein comprises SEQ ID NO: 35. In aspects of this embodiment, a F1 and/or F2 disclosed herein has an amino acid identity of, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 35. In yet other aspects of this embodiment, a F1 and/or F2 disclosed herein has an amino acid identity in the range of, e.g., about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to SEQ ID NO: 35.

In other aspects of this embodiment, a F1 and/or F2 disclosed herein has, e.g., at least 1, at least 2, at least 3, at least 4, at least 5, at least 6, at least 7, at least 8, at least 9, or at least 10 contiguous amino acid deletions, additions, and/or substitutions relative SEQ ID NO: 35; or at most 1, at most 2, at most 3, at most 4, at most 5, at most 6, at most 7, at most 8, at most 9, or at most 10 contiguous amino acid deletions, additions, and/or substitutions relative SEQ ID NO: 35. In yet other aspects of this embodiment, a F1 and/or F2 disclosed herein has, e.g., at least 1, at least 2, at least 3, at least 4, at least 5, at least 6, at least 7, at least 8, at least 9, or at least 10 non-contiguous amino acid deletions, additions, and/or substitutions relative to SEQ ID NO: 35; at most 1, at most 2, at most 3, at most 4, at most 5, at most 6, at most 7, at most 8, at most 9, or at most 10 non-contiguous amino acid deletions, additions, and/or substitutions relative to SEQ ID NO: 35.

The present fusion peptides may include a linker. In one embodiment, the linker is a peptide that ranges from about 6 to about 30 amino acids in length. In aspects of this embodiment, the peptide linker can be, e.g., at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29 or at least 30 amino acids in length. In other aspects of this embodiment, the peptide linker can be, e.g., at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29 or at most 30 amino acids in length. In other aspects of this embodiment, the peptide linker can be, e.g., about 6 to about 8, about 6 to about 10, about 6 to about 12, about 6 to about 14, about 6 to about 16, about 6 to about 18, about 6 to about 20, about 6 to about 22, about 6 to about 24, about 6 to about 26, about 6 to about 28, about 6 to about 30, about 8 to about 10, about 8 to about 12, about 8 to about 14, about 8 to about 16, about 8 to about 18, about 8 to about 20, about 8 to about 22, about 8 to about 24, about 8 to about 26, about 8 to about 28, about 8 to about 30, about 10 to about 12, about 10 to about 14, about 10 to about 16, about 10 to about 18, about 10 to about 20, about 10 to about 22, about 10 to about 24, about 10 to about 26, about 10 to about 28, about 10 to about 30, about 12 to about 14, about 12 to about 16, about 12 to about 18, about 12 to about 20, about 12 to about 22, about 12 to about 24, about 12 to about 26, about 12 to about 28, about 12 to about 30, about 14 to about 16, about 14 to about 18, about 14 to about 20, about 14 to about 22, about 14 to about 24, about 14 to about 26, about 14 to about 28, about 14 to about 30, about 16 to about 18, about 16 to about 20, about 16 to about 22, about 16 to about 24, about 16 to about 26, about 16 to about 28, about 16 to about 30, about 18 to about 20, about 18 to about 22, about 18 to about 24, about 18 to about 26, about 18 to about 28, about 18 to about 30, about 20 to about 22, about 20 to about 24, about 20 to about 26, about 20 to about 28, about 20 to about 30, about 22 to about 24, about 22 to about 26, about 22 to about 28, about 22 to about 30, about 24 to about 26, about 24 to about 28, about 24 to about 30, about 26 to about 28, about 26 to about 30 or about 26 to about 30 amino acids in length.

In an aspect of this embodiment, the linker has the amino acid sequence (GGGGS)ₙ, wherein n=2, 3 or 4 (SEQ ID NO: 1).

In yet another embodiment, a monomer of the GIP analogue containing a Fc fusion protein includes the amino acid sequence: YGEGTFISDYSIAMDKIHQQDFVNWLLAQKGGGGGSGGGGGGGGGSAESK YGPPCPPCPAPEAAGGPSVFLFPPKPKDXLM ISRTPEVTCVVVDVSQEDPEVQFNWYVDGVEVHNAKTK PREEQFNSTYRVVSVLTVLHQDWLNGKEYKCKVSNKGLPSSIEKTISKAKGQPREPQVYTLPPSQEEMT KNQVSLTCLVKGFYPSDIAVEWESNGQPENNYKTTPPVLDSDGSFXLYSXLTVDKSRWQEGNVFSCSVX HEALHNHYTQKSLSLSLG, wherein X at position 79 is selected from T and Q, X at position 257 is selected from L and M, X at position 234 is selected from F and L, and X at position 238 is selected from K and R (SEQ ID NO: 36)

In one embodiment, a monomer of GLP-1 containing a Fc fusion protein comprising the amino acid sequence: HGEGTFTSDVSSYLEEQAAKEFIAWLVKGGGGGGGSGGGGGGGGGSAESKYGPPCPPCP APEAAGGPSVFLFPPKPKDXLMISRTPEVTCVVVDVSQEDPEVQFNWYVDGVEVHNAKTKPREEQFNST YRVVSVLTVLHQDWLNGKEYKCKVSNKGLPSSIEKTISKAKGQPREPQVYTLPPSQEEMTKNQVSLTCLV KGFYPSDIAVEWESNGQPENNYKTTPPVLDSDGSFXLYSXLTVDKSRWQEGNVFSCSVXHEALHNHYT QKSLSLSLG, wherein X at position 79 is selected from T and Q, X at position 257 is selected from L and M, X at position 234 is selected from F and L, and X at position 238 is selected from K and R (SEQ ID NO: 37).

In one embodiment, a monomer of GLP-1 containing a Fc fusion protein is encoded by SEQ ID NO: 73 and encodes the fusion protein SEQ ID NO: 74. In one embodiment, a monomer of GLP-1 containing a Fc fusion protein is encoded by SEQ ID NO: 75 and encodes the fusion protein SEQ ID NO: 76.

The activity of the fusion protein may be measured by its activity against one or more of the GLP-1 receptor, the glucagon receptor, the GIP receptor, the leptin receptor, the DPP-IV, the Y5 receptor, the Melanin-concentrating hormone (MCH) receptor, the Y2/3 receptor, the MC3/4 receptor, the gastric/pancreatic lipase, the 5HT2c, the β3THE receptor, an Amylin receptor, Ghrelin, the neonatal Fc receptor (FcRn), and/or the Ghrelin receptor in one or more in vitro or in vivo assays recognized in the art as determining receptor activity. In one embodiment, the present fusion proteins have agonist activity. In one aspect, the present fusion proteins act as an agonist against a GLP-1 receptor, the GIP receptor, the glucagon receptor, a leptin receptor, a Y2/3 receptor, a MC3/4 receptor, a 5HT2c, a β3A receptor, an Amylin receptor in one or more in vitro or in vivo assays.

In one embodiment, the present fusion proteins have agonist activity against one or more of the GLP-1 receptor, the GIP receptor, the glucagon receptor. In one aspect, the present fusion proteins have agonist activity against at least two of the GLP-1 receptor, the glucagon receptor, and the GIP receptor.

In one embodiment, the selectivity of the fusion protein is determined by measuring the activity of the fusion protein against each receptor. The activity of the fusion protein may be compared to the activity of a naturally occurring (i.e. “native”) protein against a given receptor. For instance a relative ratio of receptor activity may be denoted as the molecule's activity against the first receptor relative to the activity obtained native first protein (from which the P1 protein was derived), divided by the molecule's activity at the second receptor relative to native second protein (from which the P2 protein was derived). In one embodiment, a fusion protein's relative ratio of receptor activity ranges from about 100 to 0.01, about 50 to 0.1, about 25 to 0.1, about 10 to 0.1, about 9 to 0.1, about 8 to 0.1, about 10 to 0.1, about 7 to 0.1, about 6 to 0.1, about 5 to 0.1, about 5 and 0.2, about 4 and 0.2, about 3 to 0.3, about 2 to 0.5, or about 1.

In a preferred embodiment, any of the dual-receptor agonists and triple-receptor agonists described above forms a dimer, preferably through an inter-chain covalent bond or bonds in the Fc domain. A refolding process may be used to produce the dimers, in which the agonist molecule is denatured and reduced, then diluted or buffer exchanged into a refolding buffer, and incubate overnight or up to 72 hours at a cold temperature (e.g. 0-8° C.). The refolding buffer optionally contains a redox pair, metal catalyst, sugar, amino acid, and/or urea.

In one embodiment, selectivity of a molecule for the one receptor (e.g., the GLP-1 receptor) versus a second receptor (e.g., the glucagon receptor) can be described as the relative ratio of activity of receptor 1/receptor 2 (e.g., GLP-1 receptor/Glucagon receptor activity). This GLP-1 receptor/glucagon receptor activity can be calculated as: (the molecule's activity at the GLP-1 receptor relative to native GLP-1, divided by the molecule's activity at the Glucagon receptor relative to native Glucagon); and selectivity of a molecule for the GLP-1 receptor versus GIP receptor can be described as the relative ratio of GLP-1 receptor/GIP receptor activity (the molecule's activity at the GLP-1 receptor relative to native GLP-1, divided by the molecule's activity at the GIP receptor relative to native GIP). For example, a molecule that exhibits 60% of the activity of native GLP-1 at the GLP-1 receptor and 60% of the activity of native Glucagon at the Glucagon receptor has a 1:1 ratio of GLP-1 receptor/Glucagon receptor activity, or a relative ratio of 1. Exemplary ratios of GLP-1/Glucagon activity include about 1:1, 1.5:1, 2:1, 3:1, 4:1, 5:1, 6:1, 7:1, 8:1, 9:1 or 10:1, or about 1:10, 1:9, 1:8, 1:7, 1:6, 1:5, 1:4, 1:3, 1:2, or 1:1.5. As an example, a GLP-1 receptor/Glucagon receptor activity ratio of 10:1 (or 10) indicates a 10-fold selectivity for the GLP-1 receptor versus the Glucagon receptor. Similarly, a GLP-1 receptor/glucagon receptor activity ratio of 1:10 (or 0.1) indicates a 10-fold selectivity for the Glucagon receptor versus the GLP-1 receptor.

In another embodiment, the molecule disclosed here is a dual GLP-1/GIP receptor co-agonist, wherein the GLP-1 receptor/GIP receptor activity ratio as described above is between 100 and 0.01, preferably between 10 and 0.1, further preferably between 3 and 0.3, further preferably between 2 and 0.5, and further preferably approximately 1.

In another embodiment, the molecule disclosed here is a dual GLP-1/Glucagon receptor co-agonist, wherein the GLP-1/Glucagon activity ratio as described above is between 100 and 0.01, preferably between 10 and 0.1, and further preferably between 3 and 0.3.

TABLE 7
Examples of Fusion Proteins with Dual Agonist Activity

| | | | Potential | Expression |
|---|---|---|---|---|
| Molecule | Peptides | Carrier | Fc mutations | Host |
| Class A | All natural | Dummy | With or | CHO |
| | | Mab | without T_{1/2} | |
| | | | extension | |
| Class B | All natural | IgG4 Fc | None, Knob-and-Hole | CHO |
| | | | and/or T_{1/2} | or E. coli |
| | | | extension | |
| Class C | Containing | IgG4 Fc | None, T_{1/2} | E. coli, |
| | one or more | | Extension and/or | chemically |
| | unnatural | | Knob-and-Hole | synthesized |
| | amino acid | | | peptide |

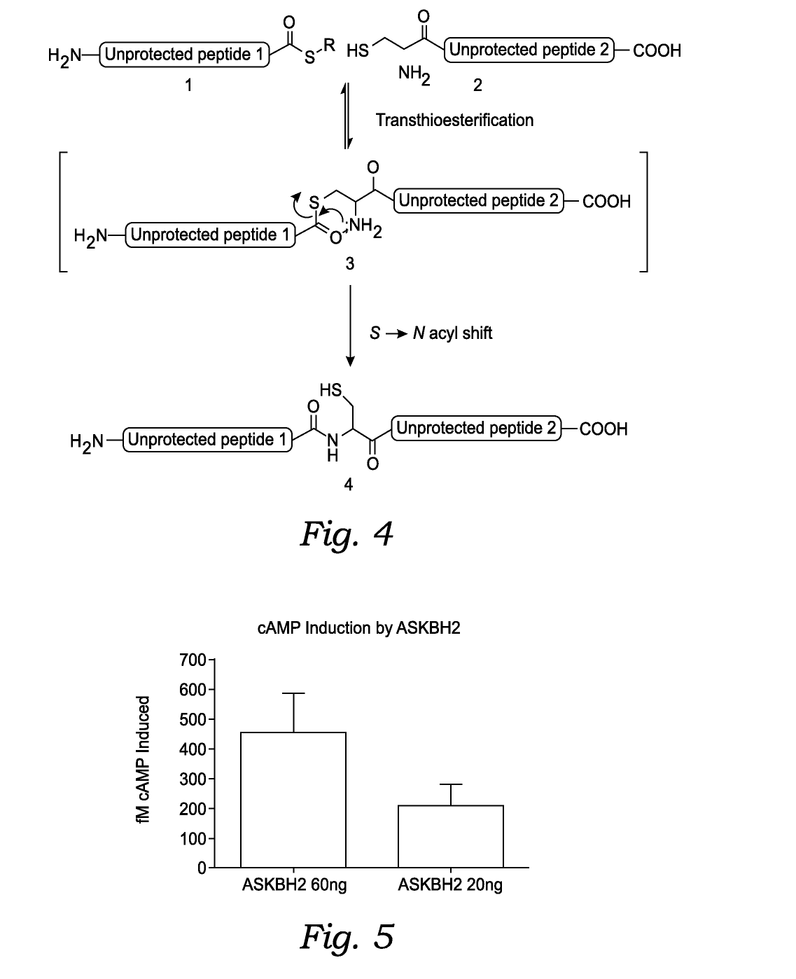

The term “native chemical ligation” (or NCL) as used herein refers to a concept for constructing a large polypeptide formed by the assembling of two or more unprotected peptides segments. Especially, NCL is the most powerful ligation method for synthesizing native backbone proteins or modified proteins. In native chemical ligation, the thiolate group of an N-terminal cysteine residue of an unprotected peptide 2 attacks the C-terminal thioester of a second unprotected peptide 1 in an aqueous buffer at suitable pH (typically) around 7, and temperature (typically 20° C.<T<37° C.). This reversible transthioesterification step is chemoselective and regioselective and leads to form a thioester intermediate 3. This intermediate rearranges by an intramolecular S,N-acyl shift that results in the formation of a native amide (‘peptide’) bond 4 at the ligation site (FIG. 4).

The term “site specific conjugation” as used herein refers to a concept where a reaction group on a chemically synthesized peptide reacts specifically to a specific group of an Fc fragment produced through the recombinant technology. For example, a peptide contains an aldehyde group can react with the 1,2-aminothiol of cysteine of a recombinant Fc fragment through site-specific thiazolidine formation, as described by Zhang and Tam, “Thiazolidine formation as a general and site-specific conjugation method for synthetic peptides and proteins.” Anal. Biochem. 1996 Jan. 1; 233(1):87-93. Such chemically synthesized peptides may contain an aldehyde group. When the Fc region is chemically synthesized, the N-terminal amino acid of the Fc analogue may be modified to Cys, allowing site specific conjugation of the P1 or P2 peptide to the Fc region.

The term “refolding” as used herein refers to the process by which a protein structure assumes its functional shape or conformation. It is the physical process by which a polypeptide folds into its characteristic and functional three-dimensional structure from random coil. It takes place at a basic pH (typically pH 8.0-10.0, pH 8.5-10, or pH 8.5-9.6), a low temperature (typically 0.0° C. to 10.0° C. or 2.0° C. to 8.0° C.), preferably with the presence of a redox pair at suitable concentrations, and/or at the presence of oxygen, and/or at the presence of catalyst(s) such as copper ions at suitable concentration.

The term “recombinant” as used herein refers to a polypeptide produced through a biological host, selected from a mammalian expression system, an insect cell expression system, a yeast expression system, and a bacterial expression system.

The term “formulation” as used herein refers to the fusion proteins disclosed herein and excipients combined together which can be administered and has the ability to bind to the corresponding receptors and initiate a signal transduction pathway resulting in the desired activity. The formulation can optionally comprise other agents so long as the fusion protein retains the ability to bind the corresponding receptors.

The term “insulinotropic activity” refers to the ability to stimulate insulin secretion in response to elevated glucose levels, thereby causing glucose uptake by cells and decreased plasma glucose levels. Insulinotropic activity may be determined in vitro and/or in vivo using methods recognized in the art. For example, insulinotropic activity can be determined by assessing the ability to stimulate insulin or inhibit glucagon secretion from the isolated perfused porcine pancreas as described in Deacon et al., “Dipeptidyl peptidase IV resistant analogues of glucagon-like peptide-1 which have extended metabolic stability and improved biological activity.” Diabetologica. 1998 March; 41(3):271-8). A GLP-1 molecule has insulinotropic activity if islet cells secrete insulin levels in the presence of the GLP-1 molecule above background levels.

The present specification also provides a pharmaceutical composition for the administration to a subject. The pharmaceutical composition disclosed herein may further include a pharmaceutically acceptable carrier, excipient, or diluent. As used herein, the term “pharmaceutically acceptable” means that the composition is sufficient to achieve the therapeutic effects without deleterious side effects, and may be readily determined depending on the type of the diseases, the patient's age, body weight, health conditions, gender, and drug sensitivity, administration route, administration mode, administration frequency, duration of treatment, drugs used in combination or coincident with the composition disclosed herein, and other factors known in medicine.

The pharmaceutical composition including the fusion protein disclosed herein may further include a pharmaceutically acceptable carrier. For oral administration, the carrier may include, but is not limited to, a binder, a lubricant, a disintegrant, an excipient, a solubilizer, a dispersing agent, a stabilizer, a suspending agent, a colorant, and a flavorant. For injectable preparations, the carrier may include a buffering agent, a preserving agent, an analgesic, a solubilizer, an isotonic agent, and a stabilizer. For preparations for topical administration, the carrier may include a base, an excipient, a lubricant, and a preserving agent.

The disclosed compositions may be formulated into a variety of dosage forms in combination with the aforementioned pharmaceutically acceptable carriers. For example, for oral administration, the pharmaceutical composition may be formulated into tablets, troches, capsules, elixirs, suspensions, syrups or wafers. For injectable preparations, the pharmaceutical composition may be formulated into an ampule as a single dosage form or a multidose container. The pharmaceutical composition may also be formulated into solutions, suspensions, tablets, pills, capsules and long-acting preparations.

On the other hand, examples of the carrier, the excipient, and the diluent suitable for the pharmaceutical formulations include, without limitation, lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate and mineral oils. In addition, the pharmaceutical formulations may further include fillers, anti-coagulating agents, lubricants, humectants, flavorants, and antiseptics.

Further, the pharmaceutical composition disclosed herein may have any formulation selected from the group consisting of tablets, pills, powders, granules, capsules, suspensions, liquids for internal use, emulsions, syrups, sterile aqueous solutions, non-aqueous solvents, lyophilized formulations and suppositories.

Further, the composition may be formulated into a single dosage form suitable for the patient's body, and preferably is formulated into a preparation useful for peptide drugs according to the typical method in the pharmaceutical field so as to be administered by an oral or parenteral route such as through skin, intravenous, intramuscular, intra-arterial, intramedullary, intramedullary, intraventricular, pulmonary, transdermal, subcutaneous, intraperitoneal, intranasal, intracolonic, topical, sublingual, vaginal, or rectal administration, but is not limited thereto.

The composition may be used by blending with a variety of pharmaceutically acceptable carriers such as physiological saline or organic solvents. In order to increase the stability or absorptivity, carbohydrates such as glucose, sucrose or dextrans, antioxidants such as ascorbic acid or glutathione, chelating agents, low molecular weight proteins or other stabilizers may be used.

The administration dose and frequency of the pharmaceutical composition disclosed herein are determined by the type of active ingredient, together with various factors such as the disease to be treated, administration route, patient's age, gender, and body weight, and disease severity.

The total effective dose of the compositions disclosed herein may be administered to a patient in a single dose, or may be administered for a long period of time in multiple doses according to a fractionated treatment protocol. In the pharmaceutical composition disclosed herein, the content of active ingredient may vary depending on the disease severity. Preferably, the total daily dose of the peptide disclosed herein may be approximately 0.0001 μg to 500 mg per 1 kg of body weight of a patient. However, the effective dose of the peptide is determined considering various factors including patient's age, body weight, health conditions, gender, disease severity, diet, and secretion rate, in addition to administration route and treatment frequency of the pharmaceutical composition. In view of this, those skilled in the art may easily determine an effective dose suitable for the particular use of the pharmaceutical composition disclosed herein. The pharmaceutical composition disclosed herein is not particularly limited to the formulation, and administration route and mode, as long as it shows suitable effects.

The pharmaceutical composition disclosed herein is expected to have longer in-vivo duration of efficacy and titer, thereby remarkably reducing the number and frequency of administration thereof.

Moreover, the pharmaceutical composition may be administered alone or in combination or coincident with other pharmaceutical formulations showing prophylactic or therapeutic effects on obesity. The pharmaceutical formulations showing prophylactic or therapeutic effects on obesity are not particularly limited, and may include a GLP-1 receptor agonist, a leptin receptor agonist, a DPP-IV inhibitor, a Y5 receptor antagonist, a Melanin-concentrating hormone (MCH) receptor antagonist, a Y2/3 receptor agonist, a MC3/4 receptor agonist, a gastric/pancreatic lipase inhibitor, a 5HT2c agonist, a β3A receptor agonist, an Amylin receptor agonist, a Ghrelin antagonist, neonatal Fc receptor (FcRn) antagonist, and/or a Ghrelin receptor antagonist.

In still another aspect, the present specification provides a method for preventing or treating of diabetes, obesity, steatosis, and related diseases comprising the step of administering to a subject the fusion protein or the pharmaceutical composition including the same. In one embodiment, the diabetes is non-insulin dependent diabetes. In one embodiment, the present compositions are applied in methods for preventing and/or treating obesity.

As used herein, the term “prevention” means all of the actions by which the occurrence of the disease is restrained or retarded. In the present specification, “prevention” means that the occurrence of diabetes or obesity from such factors as an increase in blood glucose, blood insulin levels, body weight or body fat is restrained or retarded by administration of the fusion proteins disclosed herein.

As used herein, the term “treatment” means all of the actions by which the symptoms of the disease have been alleviated, improved or ameliorated. In the present specification, “treatment” means that the symptoms of steatosis, diabetes and/or obesity are alleviated, improved or ameliorated by administration of the fusion proteins disclosed herein. For instance, in one embodiment, the treatment results in a reduction in body weight or body fat or results in insulin sensitization.

As used herein, the term “obesity” implies accumulation of an excess amount of adipose tissue in the body, and a human subject having a body mass index (body weight (kg) divided by the square of the height (m)) above 25 is to be regarded as obese. Obesity is usually caused by an energy imbalance, when the amount of dietary intake exceeds the amount of energy expended for a long period of time. Obesity is a metabolic disease that affects the whole body, and increases the risk for diabetes, hyperlipidemia, sexual dysfunction, arthritis, and cardiovascular diseases, and in some cases, is associated with incidence of cancer.

As used herein, the term “administration” means introduction of an amount of a predetermined substance into a patient by a certain suitable method. The composition disclosed herein may be administered via any of the common routes, as long as it is able to reach a desired tissue, for example, but is not limited to, intraperitoneal, intravenous, intramuscular, subcutaneous, intradermal, oral, topical, intranasal, intrapulmonary, or intrarectal administration. However, since peptides are digested upon oral administration, active ingredients of a composition for oral administration should be coated or formulated for protection against degradation in the stomach.

In the present specification, the term “subject” is those suspected of having obesity, diabetes, steatosis, and related diseases, which means mammals including human, mouse, and livestock having obesity or having the possibility of obesity. However, any subject to be treated with the fusion proteins or the pharmaceutical composition disclosed herein is included without limitation. The pharmaceutical composition including the fusion peptide disclosed herein is administered to a subject suspected of having obesity, thereby treating the subject effectively. The obesity is as described above.

The therapeutic method of the present specification may include the step of administering the composition including the fusion protein at a pharmaceutically effective amount. The total daily dose should be determined through appropriate medical judgment by a physician, and administered once or several times. The specific therapeutically effective dose level for any particular patient may vary depending on various factors well known in the medical art, including the kind and degree of the response to be achieved, concrete compositions according to whether other agents are used therewith or not, the patient's age, body weight, health condition, gender, and diet, the time and route of administration, the secretion rate of the composition, the time period of therapy, other drugs used in combination or coincident with the composition disclosed herein, and like factors well known in the medical arts.

In still another aspect, the present specification provides a use of the therapeutic protein or the pharmaceutical composition including the same in the preparation of drugs for the prevention or treatment of diabetes, obesity, steatosis, and related diseases.

In one embodiment, the dose of the composition may be administered daily, semi-weekly, weekly, bi-weekly, or monthly. The period of treatment may be for a week, two weeks, a month, two months, four months, six months, eight months, a year, or longer. The initial dose may be larger than a sustaining dose. In one embodiment, the dose ranges from a weekly dose of at least 0.01 mg, at least 0.25 mg, at least 0.3 mg, at least 0.5 mg, at least 0.75 mg, at least 1 mg, at least 1.25 mg, at least 1.5 mg, at least 2 mg, at least 2.5 mg, at least 3 mg, at least 4 mg, at least 5 mg, at least 6 mg, at least 7 mg, at least 8 mg, at least 9 mg, at least 10 mg, at least 15 mg, at least 20 mg, at least 25 mg, at least 30 mg, at least 35 mg, at least 40 mg, at least 50 mg, at least 55 mg, at least 60 mg, at least 65 mg, or at least 70 mg. In one embodiment, a weekly dose may be at most 0.5 mg, at most 0.75 mg, at most 1 mg, at most 1.25 mg, at most 1.5 mg, at most 2 mg, at most 2.5 mg, at most 3 mg, at most 4 mg, at most 5 mg, at most 6 mg, at most 7 mg, at most 8 mg, at most 9 mg, at most 10 mg, at most 15 mg, at most 20 mg, at most 25 mg, at most 30 mg, at most 35 mg, at most 40 mg, at most 50 mg, at most 55 mg, at most 60 mg, at most 65 mg, or at most 70 mg. In a particular aspect, the weekly dose may range from 0.25 mg to 2.0 mg, from 0.5 mg to 1.75 mg. In an alternative aspect, the weekly dose may range from 10 mg to 70 mg.

Aspects of the present specification can also be described as follows:
1. A dimeric fusion protein, comprising, consisting essentially of or consisting of two polypeptides, the first polypeptide comprising, consisting essentially of or consisting of a first peptide (P1) a linker (L1) and an Fc region (F1), the second polypeptide comprising, consisting essentially of or consisting of a second peptide (P2), a linker (L2) and a Fc region (F2), wherein P1 and P2 are each independently a GLP-1, a GLP-1 analogue, a glucagon, a glucacon analogue, a GIP, a GIP analogue, an oxyntomodulin, an oxyntomodulin analogue, an exendin or an exendin analogue; wherein F1 and F2 are each independently an IgG Fc, an IgA Fc, an IgM Fc, an IgD Fc, an IgE Fc, SEQ ID NO: 35 or an analogue thereof; and wherein the C-terminals of the peptides P1 and P2 are linked, though the Linkers L1 and L2, to the N-terminals of the Fc regions F1 and F2.
2. The dimeric fusion protein according to embodiment 1, wherein the GLP-1 is SEQ ID NO: 3, SEQ ID NO: 4, SEQ ID NO: 5, SEQ ID NO: 6, SEQ ID NO: 7 or SEQ ID NO: 8.
3. The dimeric fusion protein according to embodiment 1 or embodiment 2, wherein the GLP-1 analogue is SEQ ID NO: 14, SEQ ID NO: 15, SEQ ID NO: 16, SEQ ID NO: 17, SEQ ID NO: 18 or SEQ ID NO: 19, an amino acid sequence having an identity of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 3, SEQ ID NO: 4, SEQ ID NO: 5, SEQ ID NO: 6, SEQ ID NO: 7, SEQ ID NO: 8, SEQ ID NO: 14, SEQ ID NO: 15, SEQ ID NO: 16, SEQ ID NO: 17, SEQ ID NO: 18 or SEQ ID NO: 19, an amino acid sequence having at least 1, at least 2, at least 3, at least 4, or at least 5 contiguous amino acid deletions, additions, and/or substitutions relative to SEQ ID NO: 3, SEQ ID NO: 4, SEQ ID NO: 5, SEQ ID NO: 6, SEQ ID NO: 7, SEQ ID NO: 8, SEQ ID NO: 14, SEQ ID NO: 15, SEQ ID NO: 16, SEQ ID NO: 17, SEQ ID NO: 18 or SEQ ID NO: 19, an amino acid sequence having at most 1, at most 2, at most 3, at most 4, or at most 5 contiguous amino acid deletions, additions, and/or substitutions relative to SEQ ID NO: 3, SEQ ID NO: 4, SEQ ID NO: 5, SEQ ID NO: 6, SEQ ID NO: 7, SEQ ID NO: 8, SEQ ID NO: 14, SEQ ID NO: 15, SEQ ID NO: 16, SEQ ID NO: 17, SEQ ID NO: 18 or SEQ ID NO: 19, an amino acid sequence having at least 1, at least 2, at least 3, at least 4, or at least 5 non-contiguous amino acid deletions, additions, and/or substitutions relative to SEQ ID NO: 3, SEQ ID NO: 4, SEQ ID NO: 5, SEQ ID NO: 6, SEQ ID NO: 7, SEQ ID NO: 8, SEQ ID NO: 14, SEQ ID NO: 15, SEQ ID NO: 16, SEQ ID NO: 17, SEQ ID NO: 18 or SEQ ID NO: 19, or an amino acid sequence having at most 1, at most 2, at most 3, at most 4, or at most 5 non-contiguous amino acid deletions, additions, and/or substitutions relative to SEQ ID NO: 3, SEQ ID NO: 4, SEQ ID NO: 5, SEQ ID NO: 6, SEQ ID NO: 7, SEQ ID NO: 8, SEQ ID NO: 14, SEQ ID NO: 15, SEQ ID NO: 16, SEQ ID NO: 17, SEQ ID NO: 18 or SEQ ID NO: 19.
4. The dimeric fusion protein according to any one of embodiments 1-3, wherein the GIP is SEQ ID NO: 9.
5. The dimeric fusion protein according to any one of embodiments 1-4, wherein the GIP analogue is SEQ ID NO: 20 or SEQ ID NO: 21, an amino acid sequence having an identity of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 9, SEQ ID NO: 20 or SEQ ID NO: 21, an amino acid sequence having at least 1, at least 2, at least 3, at least 4, or at least 5 contiguous amino acid deletions, additions, and/or substitutions relative to SEQ ID NO: 9, SEQ ID NO: 20 or SEQ ID NO: 21, an amino acid sequence having at most 1, at most 2, at most 3, at most 4, or at most 5 contiguous amino acid deletions, additions, and/or substitutions relative to SEQ ID NO: 9, SEQ ID NO: 20 or SEQ ID NO: 21, an amino acid sequence having at least 1, at least 2, at least 3, at least 4, or at least 5 non-contiguous amino acid deletions, additions, and/or substitutions relative to SEQ ID NO: 9, SEQ ID NO: 20 or SEQ ID NO: 21, or an amino acid sequence having at most 1, at most 2, at most 3, at most 4, or at most 5 non-contiguous amino acid deletions, additions, and/or substitutions relative to SEQ ID NO: 9, SEQ ID NO: 20 or SEQ ID NO: 21.
6. The dimeric fusion protein according to any one of embodiments 1-5, wherein the exendin is an exendin-3 or an exendin-4.
7. The dimeric fusion protein according to any one of embodiments 1-6, wherein the exendin is SEQ ID NO: 10 or SEQ ID NO: 11.
8. The dimeric fusion protein according to any one of embodiments 1-7, wherein the exendin analogue is SEQ ID NO: 22, an amino acid sequence having an identity of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 10, SEQ ID NO: 11 or SEQ ID NO: 22, an amino acid sequence having at least 1, at least 2, at least 3, at least 4, or at least 5 contiguous amino acid deletions, additions, and/or substitutions relative to SEQ ID NO: 10, SEQ ID NO: 11 or SEQ ID NO: 22, an amino acid sequence having at most 1, at most 2, at most 3, at most 4, or at most 5 contiguous amino acid deletions, additions, and/or substitutions relative to SEQ ID NO: 10, SEQ ID NO: 11 or SEQ ID NO: 22, an amino acid sequence having at least 1, at least 2, at least 3, at least 4, or at least 5 non-contiguous amino acid deletions, additions, and/or substitutions relative to SEQ ID NO: 10, SEQ ID NO: 11 or SEQ ID NO: 22, or an amino acid sequence having at most 1, at most 2, at most 3, at most 4, or at most 5 non-contiguous amino acid deletions, additions, and/or substitutions relative to SEQ ID NO: 10, SEQ ID NO: 11 or SEQ ID NO: 22.
9. The dimeric fusion protein according to any one of embodiments 1-8, wherein the Glucagon is SEQ ID NO: 12.
10. The dimeric fusion protein according to any one of embodiments 1-9, wherein the Glucagon analogue is an amino acid sequence having an identity of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 12, an amino acid sequence having at least 1, at least 2, at least 3, at least 4, or at least 5 contiguous amino acid deletions, additions, and/or substitutions relative to SEQ ID NO: 12, an amino acid sequence having at most 1, at most 2, at most 3, at most 4, or at most 5 contiguous amino acid deletions, additions, and/or substitutions relative to SEQ ID NO: 12, an amino acid sequence having at least 1, at least 2, at least 3, at least 4, or at least 5 non-contiguous amino acid deletions, additions, and/or substitutions relative to SEQ ID NO: 12, or an amino acid sequence having at most 1, at most 2, at most 3, at most 4, or at most 5 non-contiguous amino acid deletions, additions, and/or substitutions relative to SEQ ID NO: 12.
11. The dimeric fusion protein according to any one of embodiments 1-10, wherein the Oxyntomodulin is SEQ ID NO: 13.
12. The dimeric fusion protein according to any one of embodiments 1-11, wherein the Oxyntomodulin analogue is SEQ ID NO: 23, SEQ ID NO: 24, SEQ ID NO: 25, SEQ ID NO: 26, SEQ ID NO: 27, SEQ ID NO: 28, SEQ ID NO: 29 or SEQ ID NO: 30, an amino acid sequence having an identity of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 13, SEQ ID NO: 23, SEQ ID NO: 24, SEQ ID NO: 25, SEQ ID NO: 26, SEQ ID NO: 27, SEQ ID NO: 28, SEQ ID NO: 29 or SEQ ID NO: 30, an amino acid sequence having at least 1, at least 2, at least 3, at least 4, or at least 5 contiguous amino acid deletions, additions, and/or substitutions relative to SEQ ID NO: 13, SEQ ID NO: 23, SEQ ID NO: 24, SEQ ID NO: 25, SEQ ID NO: 26, SEQ ID NO: 27, SEQ ID NO: 28, SEQ ID NO: 29 or SEQ ID NO: 30, an amino acid sequence having at most 1, at most 2, at most 3, at most 4, or at most 5 contiguous amino acid deletions, additions, and/or substitutions relative to SEQ ID NO: 13, SEQ ID NO: 23, SEQ ID NO: 24, SEQ ID NO: 25, SEQ ID NO: 26, SEQ ID NO: 27, SEQ ID NO: 28, SEQ ID NO: 29 or SEQ ID NO: 30, an amino acid sequence having at least 1, at least 2, at least 3, at least 4, or at least 5 non-contiguous amino acid deletions, additions, and/or substitutions relative to SEQ ID NO: 13, SEQ ID NO: 23, SEQ ID NO: 24, SEQ ID NO: 25, SEQ ID NO: 26, SEQ ID NO: 27, SEQ ID NO: 28, SEQ ID NO: 29 or SEQ ID NO: 30, or an amino acid sequence having at most 1, at most 2, at most 3, at most 4, or at most 5 non-contiguous amino acid deletions, additions, and/or substitutions relative to SEQ ID NO: 13, SEQ ID NO: 23, SEQ ID NO: 24, SEQ ID NO: 25, SEQ ID NO: 26, SEQ ID NO: 27, SEQ ID NO: 28, SEQ ID NO: 29 or SEQ ID NO: 30.
13. The dimeric fusion protein according to any one of embodiments 1-12, wherein the GLP-1, the GLP-1 analogue, the glucagon, the glucacon analogue, the GIP, the GIP analogue, the oxyntomodulin, the oxyntomodulin analogue, the exendin and/or the exendin analogue has an N-terminal amino acid of His or dHis added or substituted for the naturally occurring N-terminal amino acid.
14. The dimeric fusion protein according to any one of embodiments 1-13, wherein the second amino acid from the N-terminal of the GLP-1, the GLP-1 analogue, the glucagon, the glucacon analogue, the GIP, the GIP analogue, the oxyntomodulin, the oxyntomodulin analogue, the exendin and/or the exendin analogue is Gly, aminoisobutyric acid (Aib), D-Ala, D-Ser, D-Gly, or D-Val.
15. The dimeric fusion protein according to any one of embodiments 1-14, wherein the P1 and P2 sequences are each independently selected from the group consisting of any of SEQ ID NOs: 3-30, and an amino acid sequence having one, two, three, four, or five amino acid substitutions when compared to one or more of SEQ ID NOs: 3-30.
16. The dimeric fusion protein according to any one of embodiments 1-14, wherein the P1 and P2 sequences are each independently selected from any of SEQ ID NOs: 3-30, and an amino acid sequence having at least 80%, at least 85%, at least 90%, at least 95%, at least 97%, or at least 99% sequence identity to one or more of SEQ ID NOs: 3-30.
17. The dimeric fusion protein according to any one of embodiments 1-16, wherein P1 is a GLP-1 analogue, wherein the N-terminal of the GLP-1 analogue is either His or D-His; wherein the second amino acid of the GLP-1 analogue is selected from Gly, aminoisobutyric acid (Aib), D-Ala, D-Ser, D-Gly, and D-Val; and wherein P2 is selected from GIP, a GIP analog, Glucagon, and a Glucagon analogue.
18. The dimeric fusion protein according to any one of embodiments 1-17, wherein either P1 or P2 is a GLP-1 analogue comprising, consisting essentially of or consisting of the amino acid sequence selected from the group consisting of SEQ ID NOs: 14-19 and an amino acid sequence having one, two, three, four or five, amino acid substitutions, additions, or deletions when compared to one or more of the amino acid sequences of SEQ ID NOs: 14-19.
19. The dimeric fusion protein according to any one of embodiments 1-17, wherein either P1 or P2 is a GLP-1 analogue comprising, consisting essentially of or consisting of the amino acid sequence selected from the group consisting of SEQ ID NOs: 14-19 and an amino acid sequence having at least 80%, at least 85%, at least 90%, at least 95%, at least 97%, or at least 99% sequence identity to one or more of SEQ ID NOs: 14-19.
20. The dimeric fusion protein according to any one of embodiments 1-19, having dual agonist activity against any two of the GLP-1 receptor, the GIP receptor, and the Glucagon receptor.
21. The dimeric fusion protein according to any one of embodiments 1-20, having agonist activity against the GLP-1 receptor, the GIP receptor, and the Glucagon receptor.
22. The dimeric fusion protein according to any one of embodiments 1-21, wherein the dual receptor agonist or triple receptor agonist fusion protein comprises, consists essentially of or consists of a GIP analogue, wherein the GIP analogue comprises, consists essentially of or consists of an amino acid sequence selected from SEQ ID NOs: 20 and 21, and an amino acid sequence having one, two, three, four, or five amino acid substitutions when compared to one or more of SEQ ID NOs: 20 or 21.
23. The dimeric fusion protein according to any one of embodiments 1-22, wherein the dual receptor agonist or triple receptor agonist fusion protein comprises, consists essentially of or consists of a GIP analogue, wherein the GIP analogue comprises, consists essentially of or consists of an amino acid sequence selected from the group consisting of SEQ ID NOs: 20 and 21, and an amino acid sequence having at least 80%, at least 85%, at least 90%, at least 95%, at least 97%, or at least 99% sequence identity to one or more of SEQ ID NOs: 20 or 21.
24. The dimeric fusion protein according to any one of embodiments 1-23, wherein the IgG Fc is IgG1 Fc, an IgG2 Fc, an IgG4 Fc, or analogue thereof.
25. The dimeric fusion protein according to any one of embodiments 1-24, wherein the IgG4 Fc is SEQ ID NO: 31, SEQ ID NO: 42, SEQ ID NO: 43, SEQ ID NO: 44, SEQ ID NO: 45, SEQ ID NO: 46 or SEQ ID NO: 47.
26. The dimeric fusion protein according to any one of embodiments 1-25, wherein the IgG4 Fc analogue is an amino acid sequence having an identity of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 31, SEQ ID NO: 42, SEQ ID NO: 43, SEQ ID NO: 44, SEQ ID NO: 45, SEQ ID NO: 46 or SEQ ID NO: 47, an amino acid sequence having at least 1, at least 2, at least 3, at least 4, at least 5, at least 6, at least 7, at least 8, at least 9, or at least 10 contiguous amino acid deletions, additions, and/or substitutions relative to SEQ ID NO: 31, SEQ ID NO: 42, SEQ ID NO: 43, SEQ ID NO: 44, SEQ ID NO: 45, SEQ ID NO: 46 or SEQ ID NO: 47, an amino acid sequence having at most 1, at most 2, at most 3, at most 4, at most 5, at most 6, at most 7, at most 8, at most 9, or at most 10 contiguous amino acid deletions, additions, and/or substitutions relative to SEQ ID NO: 31, SEQ ID NO: 42, SEQ ID NO: 43, SEQ ID NO: 44, SEQ ID NO: 45, SEQ ID NO: 46 or SEQ ID NO: 47, an amino acid sequence having at least 1, at least 2, at least 3, at least 4, at least 5, at least 6, at least 7, at least 8, at least 9, or at least 10 non-contiguous amino acid deletions, additions, and/or substitutions relative to SEQ ID NO: 31, SEQ ID NO: 42, SEQ ID NO: 43, SEQ ID NO: 44, SEQ ID NO: 45, SEQ ID NO: 46 or SEQ ID NO: 47, or an amino acid sequence having at most 1, at most 2, at most 3, at most 4, at most 5, at most 6, at most 7, at most 8, at most 9, or at most 10 non-contiguous amino acid deletions, additions, and/or substitutions relative to SEQ ID NO: 31, SEQ ID NO: 42, SEQ ID NO: 43, SEQ ID NO: 44, SEQ ID NO: 45, SEQ ID NO: 46 or SEQ ID NO: 47.
27. The dimeric fusion protein according to any one of embodiments 1-26, wherein the IgG1 Fc is SEQ ID NO: 32, SEQ ID NO: 48, SEQ ID NO: 49, SEQ ID NO: 50, SEQ ID NO: 51, SEQ ID NO: 52 or SEQ ID NO: 53.
28. The dimeric fusion protein according to any one of embodiments 1-27, wherein the IgG1 Fc analogue is an amino acid sequence having an identity of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 32, SEQ ID NO: 48, SEQ ID NO: 49, SEQ ID NO: 50, SEQ ID NO: 51, SEQ ID NO: 52 or SEQ ID NO: 53, an amino acid sequence having at least 1, at least 2, at least 3, at least 4, at least 5, at least 6, at least 7, at least 8, at least 9, or at least 10 contiguous amino acid deletions, additions, and/or substitutions relative to SEQ ID NO: 32, SEQ ID NO: 48, SEQ ID NO: 49, SEQ ID NO: 50, SEQ ID NO: 51, SEQ ID NO: 52 or SEQ ID NO: 53, an amino acid sequence having at most 1, at most 2, at most 3, at most 4, at most 5, at most 6, at most 7, at most 8, at most 9, or at most 10 contiguous amino acid deletions, additions, and/or substitutions relative to SEQ ID NO: 32, SEQ ID NO: 48, SEQ ID NO: 49, SEQ ID NO: 50, SEQ ID NO: 51, SEQ ID NO: 52 or SEQ ID NO: 53, an amino acid sequence having at least 1, at least 2, at least 3, at least 4, at least 5, at least 6, at least 7, at least 8, at least 9, or at least 10 non-contiguous amino acid deletions, additions, and/or substitutions relative to SEQ ID NO: 32, SEQ ID NO: 48, SEQ ID NO: 49, SEQ ID NO: 50, SEQ ID NO: 51, SEQ ID NO: 52 or SEQ ID NO: 53, or an amino acid sequence having at most 1, at most 2, at most 3, at most 4, at most 5, at most 6, at most 7, at most 8, at most 9, or at most 10 non-contiguous amino acid deletions, additions, and/or substitutions relative to SEQ ID NO: 32, SEQ ID NO: 48, SEQ ID NO: 49, SEQ ID NO: 50, SEQ ID NO: 51, SEQ ID NO: 52 or SEQ ID NO: 53.
29. The dimeric fusion protein according to any one of embodiments 1-28, wherein the IgG2 Fc is SEQ ID NO: 33, SEQ ID NO: 54, SEQ ID NO: 55, SEQ ID NO: 56, SEQ ID NO: 57, SEQ ID NO: 58 or SEQ ID NO: 59.
30. The dimeric fusion protein according to any one of embodiments 1-29, wherein the IgG2 Fc analogue is an amino acid sequence having an identity of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 33, SEQ ID NO: 54, SEQ ID NO: 55, SEQ ID NO: 56, SEQ ID NO: 57, SEQ ID NO: 58 or SEQ ID NO: 59, an amino acid sequence having at least 1, at least 2, at least 3, at least 4, at least 5, at least 6, at least 7, at least 8, at least 9, or at least 10 contiguous amino acid deletions, additions, and/or substitutions relative to SEQ ID NO: 33, SEQ ID NO: 54, SEQ ID NO: 55, SEQ ID NO: 56, SEQ ID NO: 57, SEQ ID NO: 58 or SEQ ID NO: 59, an amino acid sequence having at most 1, at most 2, at most 3, at most 4, at most 5, at most 6, at most 7, at most 8, at most 9, or at most 10 contiguous amino acid deletions, additions, and/or substitutions relative to SEQ ID NO: 33, SEQ ID NO: 54, SEQ ID NO: 55, SEQ ID NO: 56, SEQ ID NO: 57, SEQ ID NO: 58 or SEQ ID NO: 59, an amino acid sequence having at least 1, at least 2, at least 3, at least 4, at least 5, at least 6, at least 7, at least 8, at least 9, or at least 10 non-contiguous amino acid deletions, additions, and/or substitutions relative to SEQ ID NO: 33, SEQ ID NO: 54, SEQ ID NO: 55, SEQ ID NO: 56, SEQ ID NO: 57, SEQ ID NO: 58 or SEQ ID NO: 59, or an amino acid sequence having at most 1, at most 2, at most 3, at most 4, at most 5, at most 6, at most 7, at most 8, at most 9, or at most 10 non-contiguous amino acid deletions, additions, and/or substitutions relative to SEQ ID NO: 33, SEQ ID NO: 54, SEQ ID NO: 55, SEQ ID NO: 56, SEQ ID NO: 57, SEQ ID NO: 58 or SEQ ID NO: 59.
31. The dimeric fusion protein according to any one of embodiments 1-30, wherein the IgG2 Fc is SEQ ID NO: 34, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71 or SEQ ID NO: 72.
32. The dimeric fusion protein according to any one of embodiments 1-31, wherein the IgG2 Fc analogue is an amino acid sequence having an identity of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 34, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71 or SEQ ID NO: 72, an amino acid sequence having at least 1, at least 2, at least 3, at least 4, at least 5, at least 6, at least 7, at least 8, at least 9, or at least 10 contiguous amino acid deletions, additions, and/or substitutions relative to SEQ ID NO: 34, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71 or SEQ ID NO: 72, an amino acid sequence having at most 1, at most 2, at most 3, at most 4, at most 5, at most 6, at most 7, at most 8, at most 9, or at most 10 contiguous amino acid deletions, additions, and/or substitutions relative to SEQ ID NO: 34, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71 or SEQ ID NO: 72, an amino acid sequence having at least 1, at least 2, at least 3, at least 4, at least 5, at least 6, at least 7, at least 8, at least 9, or at least 10 non-contiguous amino acid deletions, additions, and/or substitutions relative to SEQ ID NO: 34, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71 or SEQ ID NO: 72, or an amino acid sequence having at most 1, at most 2, at most 3, at most 4, at most 5, at most 6, at most 7, at most 8, at most 9, or at most 10 non-contiguous amino acid deletions, additions, and/or substitutions relative to SEQ ID NO: 34, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71 or SEQ ID NO: 72.
33. The dimeric fusion protein according to any one of embodiments 1-32, wherein the SEQ ID NO: 35 analogue is an amino acid sequence having an identity of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, to SEQ ID NO: 35, an amino acid sequence having at least 1, at least 2, at least 3, at least 4, at least 5, at least 6, at least 7, at least 8, at least 9, or at least 10 contiguous amino acid deletions, additions, and/or substitutions relative to SEQ ID NO: 35, an amino acid sequence having at most 1, at most 2, at most 3, at most 4, at most 5, at most 6, at most 7, at most 8, at most 9, or at most 10 contiguous amino acid deletions, additions, and/or substitutions relative to SEQ ID NO: 35, an amino acid sequence having at least 1, at least 2, at least 3, at least 4, at least 5, at least 6, at least 7, at least 8, at least 9, or at least 10 non-contiguous amino acid deletions, additions, and/or substitutions relative to SEQ ID NO: 35, or an amino acid sequence having at most 1, at most 2, at most 3, at most 4, at most 5, at most 6, at most 7, at most 8, at most 9, or at most 10 non-contiguous amino acid deletions, additions, and/or substitutions relative to SEQ ID NO: 35.
34. The dimeric fusion protein according to any one of embodiments 1-33, wherein the dual receptor agonist or triple receptor agonist fusion protein comprises, consists essentially of or consists of an IgG Fc analogue, an IgA Fc analogue, an IgM Fc analogue, and/or an IgE Fc analogue.
35. The dimeric fusion protein according to any one of embodiments 1-34, comprising, consisting essentially of or consisting of an IgG4, IgG2, IgG1, or IgM Fc analogue.
36. The dimeric fusion protein according to any one of embodiments 1-35, comprising, consisting essentially of or consisting of an IgG4 Fc analogue.
37. The dimeric fusion protein according to any one of embodiments 1-36, wherein the first polypeptide, the second polypeptide, or both comprise an IgG4 Fc analogue, wherein F1 or F2 comprises, consists essentially of or consists of an amino acid sequence selected from any of SEQ ID NOs: 31-35 and 42-71, and an amino acid sequence having one, two, three, four or five, amino acid substitutions, additions, or deletions when compared to one or more of SEQ ID NOs: 31-35 and 42-71.
38. The dimeric fusion protein according to any one of embodiments 1-36, wherein the first polypeptide, the second polypeptide, or both comprise an IgG4 Fc analogue, wherein F1 or F2 comprises, consists essentially of or consists of an amino acid sequence selected from any of SEQ ID NOs: 31-35 and 42-71, and an amino acid sequence having at least 80%, at least 85%, at least 90%, at least 95%, at least 97%, or at least 99% sequence identity to one or more of SEQ ID NOs: 31-35 and 42-71.
39. The dimeric fusion protein according to any one of embodiments 1-38, wherein the first polypeptide, the second polypeptide, or both comprise a GLP-1 analogue-Fc fusion protein comprising, consisting essentially of or consisting of the amino acid sequence of SEQ ID NO: 37, or an amino acid sequence having one, two, three, four or five, amino acid substitutions, additions, or deletions when compared to SEQ ID NO: 37.
40. The dimeric fusion protein according to any one of embodiments 1-38, wherein the first polypeptide, the second polypeptide, or both comprise a GLP-1 analogue-Fc fusion protein comprising, consisting essentially of or consisting of the amino acid sequence of SEQ ID NO: 37, or an amino acid sequence having at least 80%, at least 85%, at least 90%, at least 95%, at least 97%, or at least 99% sequence identity to SEQ ID NO: 37.
41. The dimeric fusion protein according to any one of embodiments 1-38, wherein the first polypeptide, the second polypeptide, or both comprise a GIP analogue-Fc fusion protein comprising, consisting essentially of or consisting of the amino acid sequence of SEQ ID No. 36 or an amino acid sequence having one, two, three, four or five, amino acid substitutions, additions, or deletions when compared to SEQ ID NO: 36.
42. The dimeric fusion protein according to any one of embodiments 1-38, wherein the first polypeptide, the second polypeptide, or both comprise a GIP analogue-Fc fusion protein comprising, consisting essentially of or consisting of the amino acid sequence of SEQ ID No. 36, or an amino acid sequence having at least 80%, at least 85%, at least 90%, at least 95%, at least 97%, or at least 99% sequence identity to SEQ ID NO: 36.
43. The dimeric fusion protein according to any one of embodiments 1-38, wherein the first polypeptide, the second polypeptide, or both comprise is encoded by the polynucleotide SEQ ID NO: 73 or SEQ ID NO: 75 or encoded by the polynucleotide having one, two, three, four or five, amino acid substitutions, additions, or deletions when compared to SEQ ID NO: 73 or SEQ ID NO: 75.
44. The dimeric fusion protein according to any one of embodiments 1-38, wherein the first polypeptide, the second polypeptide, or both comprise is encoded by the polynucleotide SEQ ID NO: 73 or SEQ ID NO: 75 or encoded by the polynucleotide having at least 80%, at least 85%, at least 90%, at least 95%, at least 97%, or at least 99% sequence identity to SEQ ID NO: 73 or SEQ ID NO: 75.
45. The dimeric fusion protein according to any one of embodiments 1-38, wherein the first polypeptide, the second polypeptide, or both comprise SEQ ID NO: 74 or SEQ ID NO: 76 or an amino acid sequence having one, two, three, four or five, amino acid substitutions, additions, or deletions when compared to SEQ ID NO: 74 or SEQ ID NO: 76.
46. The dimeric fusion protein according to any one of embodiments 1-38, wherein the first polypeptide, the second polypeptide, or both comprise SEQ ID NO: 74 or SEQ ID NO: 76 or an amino acid sequence having at least 80%, at least 85%, at least 90%, at least 95%, at least 97%, or at least 99% sequence identity to SEQ ID NO: 74 or SEQ ID NO: 76.
47. The dimeric fusion protein according to any one of embodiments 1-46, where the fusion protein comprises, consists essentially of or consists of a unimolecular dual agonist for the GLP-1 receptor and the Glucagon receptor.
48. The dimeric fusion protein according to any one of embodiments 1-46, where the dual receptor agonist or triple receptor agonist molecule comprises, consists essentially of or consists of a unimolecular dual agonist for the GLP-1 receptor and the GIP receptor.
49. The dimeric fusion protein according to any one of embodiments 1-48, wherein an imidazolic group is attached to the N-terminal of P1 and/or P2.
50. The dimeric fusion protein according to embodiment 49, wherein the imidazolic group is selected from the group consisting of 4-imidazopropionyl (des-amino-histidyl), 4-imidzoacetyl, 5-imidazo-α, α dimethyl-acetyl.
51. The dimeric fusion protein according to embodiment 50, wherein the imidazolic group is 4-imidoacetyl.
52. The dimeric fusion protein according to any one of embodiments 1-51, wherein the fusion protein is a dual GLP-1 receptor and GIP receptor co-agonist, wherein the molecule has a relative ratio of GLP-1 receptor/GIP receptor activity in the range of 100 and 0.01.
53. The dimeric fusion protein according to any one of embodiments 1-52, wherein the fusion protein is a dual GLP-1 receptor and GIP receptor co-agonist, wherein the molecule has a relative ratio of GLP-1 receptor/GIP receptor activity in the range of 10 and 0.1.
54. The dimeric fusion protein according to any one of embodiments 1-53, wherein the fusion protein is a dual GLP-1 receptor and GIP receptor co-agonist, wherein the molecule has a relative ratio of GLP-1 receptor/GIP receptor activity in the range of 5 and 0.2.
55. The dimeric fusion protein according to any one of embodiments 1-53, wherein the fusion protein is a dual GLP-1 receptor and GIP receptor co-agonist, wherein the molecule has a relative ratio of GLP-1 receptor/GIP receptor activity in the range of 3 and 0.3.
56. The dimeric fusion protein according to any one of embodiments 1-53, wherein the fusion protein is a dual GLP-1 receptor and GIP receptor co-agonist, wherein the molecule has a relative ratio of GLP-1 receptor/GIP receptor activity in the range of 2 and 0.5.
57. The dimeric fusion protein according to any one of embodiments 1-53, wherein the fusion protein is a dual GLP-1 receptor and GIP receptor co-agonist, wherein the molecule has a relative ratio of GLP-1 receptor/GIP receptor activity of approximately 1.
58. The dimeric fusion protein according to any one of embodiments 1-53, wherein the fusion protein is a dual GLP-1 receptor and glucagon receptor co-agonist, wherein the molecule has a relative ratio of GLP-1 receptor/Glucagon receptor activity in the range of 100 and 0.01.
59. The dimeric fusion protein according to any one of embodiments 1-53, wherein the fusion protein is a dual GLP-1 receptor and glucagon receptor co-agonist, wherein the molecule has a relative ratio of GLP-1 receptor/Glucagon receptor activity in the range of 10 and 0.1.
60. The dimeric fusion protein according to any one of embodiments 1-53, wherein the fusion protein is a dual GLP-1 receptor and glucagon receptor co-agonist, wherein the molecule has a relative ratio of GLP-1 receptor/Glucagon receptor activity in the range of 5 and 0.2.
61. The dimeric fusion protein according to any one of embodiments 1-53, wherein the fusion protein is a dual GLP-1 receptor and glucagon receptor co-agonist, wherein the molecule has a relative ratio of GLP-1 receptor/Glucagon receptor in the range of 2 and 0.5.
62. The dimeric fusion protein according to any one of embodiments 1-53, wherein the fusion protein is a dual GLP-1 receptor and glucagon receptor co-agonist, wherein the molecule has a relative ratio of GLP-1 receptor/Glucagon receptor activity of approximately 1.
63. The dimeric fusion protein according to any one of embodiments 1-62, wherein the fusion protein comprises, consists essentially of or consists of an IgG Fc, wherein the IgG Fc is mutated to have increased binding affinity to neonatal Fc receptor (FcRn) compared to its wild type counterpart.
64. The dimeric fusion protein according to any one of embodiments 1-63, wherein the fusion protein comprises, consists essentially of or consists of an IgG Fc, wherein the Fc is modified to have increased binding affinity to neonatal Fc receptor (FcRn) comparing to its wild type counterpart, wherein the Fc region comprising, consisting essentially of or consisting of amino acid residues 250 and 428 that are substituted with glutamic acid or glutamine at amino acid residue 250 and leucine or phenylalanine at amino acid residue 428, wherein the amino acid residues are numbered by the EU numbering system.
65. The dimeric fusion protein according to any one of embodiments 1-64, wherein the fusion protein comprises, consists essentially of or consists of an IgG Fc, wherein the half-life of the molecule in serum is 6 days or longer.
66. The dimeric fusion protein according to any one of embodiments 1-65, wherein the Fc polypeptide F2 comprising, consisting essentially of or consisting of the cavity comprises, consists essentially of or consists of two or more amino acid replacements selected from the group consisting of T366S, L368A, and Y407V, using amino acid numbering according to the EU numbering scheme of Kabat.
67. The dimeric fusion protein according to any one of embodiments 1-66, wherein the Fc polypeptide F1 comprising, consisting essentially of or consisting of the protuberance comprises, consists essentially of or consists of replacement of threonine at position 366 with tryptophan or tyrosine, using the amino acid numbering according to the EU numbering scheme of Kabat.
68. The dimeric fusion protein according to any one of embodiments 1-67, wherein the fusion protein is a homodimer.
69. The dimeric fusion protein according to any one of embodiments 1-67, wherein the fusion protein is a heterodimer.
70. The dimeric fusion protein according to embodiment 69, wherein
   a) the first peptide P1 is linked to an IgG Fc fragment (F1) and the second peptide is fused to another IgG Fc fragment (F2)
   b) F1 and F2 are derived from the same IgG Fc, but having different amino acid sequences;
   c) F1 has an amino acid residue in the interface is replaced with an amino acid residue having a larger side chain volume, thereby generating a protuberance on F1; or
   d) residue having a smaller side chain volume, thereby generating a cavity in F2, wherein the protuberance of F1 may be positioned in the cavity upon assembly of the heterogenous dimer.
71. The dimeric fusion protein according to embodiment 69 or embodiment 70, wherein the first polypeptide comprises, consists essentially of or consists of an amino acid sequence SEQ ID No. 36 or an amino acid sequence having one, two, three, four or five, amino acid substitutions, additions, or deletions when compared to SEQ ID NO: 36, and the second polypeptide comprises, consists essentially of or consists of an amino acid sequence as shown in SEQ ID NO: 37 or an amino acid sequence having one, two, three, four or five, amino acid substitutions, additions, or deletions when compared to SEQ ID NO: 37.
72. The dimeric fusion protein according to embodiment 69 or embodiment 70, wherein the first polypeptide comprises, consists essentially of or consists of an amino acid sequence SEQ ID No. 36 or an amino acid sequence having at least 80%, 85%, 90%, 95%, 97%, or 99% sequence homology to SEQ ID NO: 36, and the second polypeptide comprises, consists essentially of or consists of an amino acid sequence as shown in SEQ ID NO: 37 or an amino acid sequence having at least 80%, at least 85%, at least 90%, at least 95%, at least 97%, or at least 99% sequence identity to SEQ ID NO: 37.
73. The dimeric fusion protein according to embodiment 69 or embodiment 70, wherein the first polypeptide comprises, consists essentially of or consists of an amino acid sequence as in SEQ ID NO: 38 or an amino acid sequence having one, two, three, four or five, amino acid substitutions, additions, or deletions when compared to SEQ ID NO: 38, and the second polypeptide comprises, consists essentially of or consists of an amino acid sequence as shown in SEQ ID NO: 39 or an amino acid sequence having one, two, three, four or five, amino acid substitutions, additions, or deletions when compared to SEQ ID NO: 39.
74. The dimeric fusion protein according to embodiment 69 or embodiment 70, wherein the first polypeptide comprises, consists essentially of or consists of an amino acid sequence as in SEQ ID NO: 38 or an amino acid sequence having at least 80%, 85%, 90%, 95%, 97%, or 99% sequence homology to SEQ ID NO: 38, and the second polypeptide comprises, consists essentially of or consists of an amino acid sequence as shown in SEQ ID NO: 39 or an amino acid sequence having at least 80%, at least 85%, at least 90%, at least 95%, at least 97%, or at least 99% sequence identity to SEQ ID NO: 39.
75. The dimeric fusion protein according to any one of embodiments 1-74, wherein at least one of the two peptides comprises, consists essentially of or consists of at least one unnatural amino acid; wherein the peptide containing unnatural amino acid is chemically synthesized; wherein the N-terminal of the recombinant Fc analogue is Cys; wherein the peptide is fused to the recombinant Fc analogue through native chemical ligation.
76. The dimeric fusion protein according to any one of embodiments 1-74, wherein at least one of the two peptides comprises, consists essentially of or consists of at least one unnatural amino acid and the peptide containing the unnatural amino acid is chemically synthesized; and the chemically synthesized peptide comprises, consists essentially of or consists of an aldehyde group; and the N-terminal of the recombinant Fc analogue is Cys; and the peptide is conjugated site-specifically to the N-terminal of the recombinant Fc analogue through thiazolidine formation.
77. The dimeric fusion protein according to any one of embodiments 1-76 for use as a medicament.
78. The dimeric fusion protein according to any one of embodiments 1-76 for use in the treatment of diabetes, obesity, or steatosis.
79. The dimeric fusion protein according to any one of embodiments 1-76 for use in the treatment of non-insulin dependent diabetes mellitus.
80. The dimeric fusion protein according to any one of embodiments 1-76 for use in the treatment of obesity or inducement of weight loss in an overweight subject.
81. A formulation comprising, consisting essentially of or consisting of dimeric fusion protein as defined in any one of embodiments 1-80.
82. A method for making a dimeric fusion protein as defined in any one of embodiments 1-80 comprising, consisting essentially of or consisting of: reacting the aldehyde group of a P1 or P2 peptide with an Fc analogue having an N-terminal cysteine by reacting the aldehyde with the 1,2-aminothiol of cysteine.
83. A pharmaceutical composition comprising, consisting essentially of or consisting of a dimeric fusion protein as defined in any one of embodiments 1-80.
84. A method of treating diabetes, steatosis, or obesity comprising, consisting essentially of or consisting of administering to a subject in need thereof a pharmaceutical composition comprising, consisting essentially of or consisting of a dimeric fusion protein as defined in any one of embodiments 1-80.
85. The method according to claim 84, wherein the composition is administered daily, semi-weekly, weekly, bi-weekly, or monthly.
86. The method according to embodiment 84 or embodiment 85 wherein the period of treatment is for about a week, two weeks, a month, two months, four months, six months, eight months, a year, two years, three years, five years, ten years, or 20 years.
87. The method according to any one of embodiments 84-86, wherein the initial dosage is larger than a sustaining dose.
88. The method according to any one of embodiments 84-87, wherein the dose ranges from a weekly dose of about at least about 0.01 mg, at least about 0.25 mg, at least about 0.3 mg, at least about 0.5 mg, at least about 0.75 mg, at least about 1 mg, at least about 1.25 mg, at least about 1.5 mg, at least about 2 mg, at least about 2.5 mg, at least about 3 mg, at least about 4 mg, at least about 5 mg, at least about 6 mg, at least about 7 mg, at least about 8 mg, at least about 9 mg, at least about 10 mg, at least about 15 mg, at least about 20 mg, at least about 25 mg, at least about 30 mg, at least about 35 mg, at least about 40 mg, at least about 50 mg, at least about 55 mg, at least about 60 mg, at least about 65 mg, or at least about 70 mg.
89. The method according to any one of embodiments 84-88, wherein the weekly dose is about at most about 0.5 mg, at most about 0.75 mg, at most about 1 mg, at most about 1.25 mg, at most about 1.5 mg, at most about 2 mg, at most about 2.5 mg, at most about 3 mg, at most about 4 mg, at most about 5 mg, at most about 6 mg, at most about 7 mg, at most about 8 mg, at most about 9 mg, at most about 10 mg, at most about 15 mg, at most about 20 mg, at most about 25 mg, at most about 30 mg, at most about 35 mg, at most about 40 mg, at most about 50 mg, at most about 55 mg, at most about 60 mg, at most about 65 mg, or at most about 70 mg. In a particular aspect, the weekly dose may range from about 0.25 mg to about 2.0 mg, from about 0.5 mg to about 1.75 mg. In an alternative aspect, the weekly dose may range from about 10 mg to about 70 mg.
90. The method according to any one of embodiments 84-89, wherein the weekly dose ranges from about 0.25 mg to about 2.0 mg or from about 0.5 mg to about 1.75 mg.
91. The method according to any one of embodiments 84-90, wherein the weekly dose ranges from about 10 mg to about 70 mg.

### EXAMPLES

The following non-limiting examples are provided for illustrative purposes only in order to facilitate a more complete understanding of the disclosed subject matter. These examples should not be construed to limit any of the embodiments described in the present specification, including those pertaining to the fusion peptides, pharmaceutical compositions, or methods and uses for treating diabetes, obesity, steatosis, and/or related diseases.

### Example 1

### Peptide Synthesis

The native chemical ligation reaction can be carried out following the procedure below:

A stock solution of 6M GuHCI (guanidine hydrochloride) and 0.1M Na₂HPO₄ (sodium phosphate dibasic . . . monobasic will work as well) is created. The GuHCI is for solubilizing the peptide reactants and the Na₂HPO₄ is to buffer the solution near pH 6.8-7.

For 50 mL of stock solution: weigh 1.42 g (10 mmol) of sodium phosphate (mono or dibasic) into a 150 mL beaker. Add about 25 mL water and dissolve. Then 28.7 g (300 mmol) of GuHCI is added and stirred until dissolved, adding more water if necessary up to about 45 mL (the GuHCI will greatly expand the volume of water). The dissolution of the GuHCI is a very endothermic process and the beaker will become very cold, and may need to be warmed for full dissolution. When the solids are completely dissolved, the solution is poured into a 50 mL volumetric flask and make up to 50 mL. The solution may be optionally transferred to a capped bottle for storage if desired. This stock solution can be stored at 4° C. for several months.

The NCL buffer solution is prepared by adding MPAA (4-mercaptophenylacetic acid) and TCEP.HCl (tris-2-carboxyethylphosphine hydrochloride). Although it is not known how long this solution can be kept once the TCEP.HCl has been added, it is common practice to use freshly prepared NCL buffer.

Filtered stock solution is subsequently added to MPAA and TCEP to make up a 50 mM (Johnson E C B, Kent S B H. Journ. Amer. Chem. Soc. 128 (2006), pp. 6640-6646) MPAA (the thiol catalyst) and 20 mM TCEP (the reducing agent) solution. To illustrate how this is done ligation on the scale of 5 mg of each peptide (about 3 mM each) may be done as follows: 5 mL stock solution is filtered using a 0.2 μm syringe-driven filter into a 20 mL scintillation vial containing 28.7 mg TCEP.HCL (0.1 mmol) and 42.1 mg MPAA (0.26 mmol). Note that MPAA has poor solubility at low pH; the solubility of MPAA is enhanced when the pH is raised to around 7.

Note: In lieu of TCEP.HCL addition, or in conjunction with it, the ligation buffer is optionally de-gassed. Whether to degas or not is up to the researcher's experimental judgment. As a general rule, it is better to avoid a problem (the presence of dissolved oxygen) than to add a chemical intended to obviate a problem; each chemical present in a reaction can, and usually does, cause side reactions at some level. TCEP is no exception . . . it has been shown to desulfurize Cys-peptides in the absence of MPAA after several hours, for example. Nevertheless, it is very useful for most NCL reactions and at 20 mM (and with 50 mM MPAA) causes few noticeable side reactions.

Minimum amounts of 2 M NaOH or 1 M HCl are added to adjust the pH of the ligation buffer to about 7.1 using a freshly calibrated pH meter; when the buffer is added to the reacting peptides, traces of TFA co-purified with the peptides will lower the pH of the solution to the desired pH 7.0.

The desired amounts of peptide-thioester and Cys-peptide are accurately weighed and added into a glass scintillation vial or plastic centrifuge tube. Amounts of each peptide are used to obtain a final concentrations between 1-5 mM, for 5 mL of NCL solution. When the peptide is small (about 1500 Da) this should come out to around 5 mg for 3 mM concentrations of each. Also, the more hydrophilic peptide (using reverse phase-LC) is preferably in slight (e.g. about 20%) excess, as chances are the product will match the hydrophobicity of the more hydrophobic peptide and will co-elute.

The buffer solution (phosphate, 6 M Gu.HCl, MPAA, TCEP.HCl) is transferred to the glass scintillation vial or plastic centrifuge tube containing the appropriate amounts of peptide-thioester and Cys-peptide. Once the peptides have dissolved, the vial or tube is capped. The pH is checked and adjusted if necessary to 7.0. The adjustment should be done carefully with a more dilute NaOH solution (we use 0.2 M NaOH). If the pH climbs very high above 7, the peptide-thioester will be hydrolyzed.

The reaction is carried out at ambient temperature (20-25° C.) without mixing in most cases.

The reaction is monitored by one of multiple preferred methods (such as LCMS or MALDI-TOF MS to follow the reaction). Aliquots are taken at “t=0” immediately after the NCL buffer is added to the peptides and then aliquots at 30 minutes, 1 h, 2 h, 4 h, 8 h, 16 h are taken. Depending on the identity of the variable amino acid at the Xaa-Cys ligation site, the reaction is finished after anywhere from an hour to 16 hours (ligations at Val, Thr, Ile, Pro can take longer).

To take an aliquot, 10 μL of solution is removed and added into a 1.5 mL plastic micro-centrifuge tube. The 10 μL solution is diluted 20 fold (190 μL) with 50:50 AcCN:H₂O (0.08%:0.1% TFA v/v). The dilution effectively stops the reaction, but to ensure that the reaction is stopped sure a few μL's of 1 M HCl can be added, while bringing the pH down to about 4. This will completely terminate the reaction, and shouldn't give rise to side reactions. The diluted aliquot should be analyzed as soon as possible.

When the reaction is complete, immediately dilute the solution to approx. 1 M GuHCI using aqueous 0.1% TFA (solvent A), which will also drop the pH, enabling loading onto the prep-HPLC (acidic injections are preferred). This will involve diluting the NCL reaction solution 6-fold with solvent A.

### Example 2

### Chemical Synthesis of the Fc Fusion Protein with Unnatural Amino Acids

Where the peptide is an Fc fusion protein, and at least one of the two peptides contains at least one unnatural amino acid, the peptide containing the unnatural amino acid is chemically synthesized. The chemically synthesized peptide can contain an aldehyde group and the N-terminal of the recombinant Fc analogue can be Cys. The peptide can be site-specifically conjugated to the N-terminal of the recombinant Fc analogue through thiazolidine formation. The site specific conjugation can be carried out as described by Zhang and Tam: “Thiazolidine formation as a general and site-specific conjugation method for synthetic peptides and proteins.” Anal Biochem 1996 Jan. 1; 233(1):87-93.)

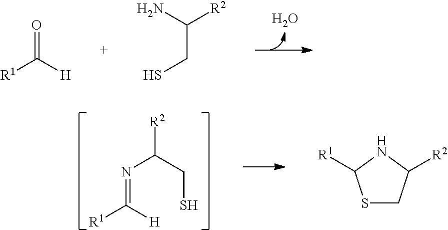

The purity of the fusion protein can be analyzed using analytical methods including RP-HPLC, IEX-HPLC, SEC-HPLC, and CIEF.

The in vitro and ex vivo activity of the fusion protein is assessed for receptor binding using a cell line expressing the cloned receptor, and for ability to stimulate insulin or inhibit glucagon secretion from the isolated perfused porcine pancreas as described in Diabetologia. 1998 March; 41(3):271-8, the content of which is hereby expressly incorporated by reference in its entirety for all purposes. Other in vitro and ex vivo methods may also be used. The in vivo activity of the fusion protein is analyzed by an animal efficacy study in mice, rats, hamsters, monkeys, pigs, sheep, or other animal models of diabetes and obesity.

### Example 3

### Fusion Peptide

After chemical ligation, a peptide having an Fc, a linker, and a P1 or P2 is obtained. For instance the peptides of the present disclosure may include one or more of the following sequences: a GLP-1 analogue containing fusion peptide of SEQ ID NO: 38; a GIP analogue containing peptide of SEQ ID NO: 39; a specific Fc region of SEQ ID NO: 40.

### Example 4

### Biological Activity Assay

Biological activity of a fusion peptide disclosed herein (designated ASKBH2) was determined using the rat insulinoma cell line RIN-m5F. Upon receptor activation by GLP-1, RIN-m5F cells show an increase in intracellular cAMP. In this assay RIN-m5F cells were incubated with various concentrations of ASKBH2 for 25 minutes, then cells were lysed and assayed for intracellular cAMP using an Assay Designs cAMP ELISA kit. Data shows cAMP induced above medium alone without GLP-1 (FIG. 5).

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

1. A dimeric fusion protein, comprising two polypeptides, the first polypeptide comprising a first peptide (P1) a linker (L1) and an Fc region (F1), the second polypeptide comprising a second peptide (P2), a linker (L2) and a Fc region (F2),
wherein P1 and P2 are each independently a GLP-1, a GLP-1 analogue, a glucagon, a glucacon analogue, a GIP, a GIP analogue, an oxyntomodulin, an oxyntomodulin analogue, an exendin or an exendin analogue;
wherein F1 and F2 are each independently an IgG Fc, an IgG Fc analogue, an IgA Fc, an IgA Fc analogue, an IgM Fc, an IgM Fc analogue, an IgD Fc, an IgD Fc analogue, an IgE Fc, an IgE Fc analogue, SEQ ID NO: 35 and/or a SEQ ID NO: 35 analogue; and
wherein the C-terminals of the peptides P1 and P2 are linked, though the Linkers L1 and L2, to the N-terminals of the Fc regions F1 and F2.

2. The dimeric fusion protein according to claim 1, wherein the GLP-1, the GLP-1 analogue, the glucagon, the glucacon analogue, the GIP, the GIP analogue, the oxyntomodulin, the oxyntomodulin analogue, the exendin and/or the exendin analogue has an N-terminal amino acid of His or dHis added or substituted for the naturally occurring N-terminal amino acid.

3. The dimeric fusion protein according to claim 1, wherein the second amino acid from the N-terminal of the GLP-1, the GLP-1 analogue, the glucagon, the glucacon analogue, the GIP, the GIP analogue, the oxyntomodulin, the oxyntomodulin analogue, the exendin and/or the exendin analogue is Gly, aminoisobutyric acid (Aib), D-Ala, D-Ser, D-Gly, or D-Val.

4. The dimeric fusion protein according to claim 1, wherein P1 is a GLP-1 analogue, wherein the N-terminal of the GLP-1 analogue is either His or D-His; wherein the second amino acid of the GLP-1 analogue is selected from Gly, aminoisobutyric acid (Aib), D-Ala, D-Ser, D-Gly, and D-Val; and wherein P2 is selected from GIP, a GIP analog, Glucagon, and a Glucagon analogue.

5. The dimeric fusion protein according to claim 1, having agonist activity against the GLP-1 receptor, the GIP receptor, and the Glucagon receptor.

6. The dimeric fusion protein according to claim 1, having dual agonist activity against any two of the GLP-1 receptor, the GIP receptor, and the Glucagon receptor.

7. The dimeric fusion protein according to claim 1, wherein the IgG Fc is IgG1 Fc, an IgG2 Fc, or an IgG4 Fc.

8. The dimeric fusion protein according to claim 1, wherein the first polypeptide, the second polypeptide, or both comprise an IgG4 Fc analogue, wherein F1 or F2 comprises an amino acid sequence selected from any of SEQ ID NOs: 31-35 and 42-71, an amino acid sequence having one, two, three, four or five, amino acid substitutions, additions, or deletions when compared to one or more of SEQ ID NOs: 31-35 and 42-71, or an amino acid sequence having at least 80%, at least 85%, at least 90%, at least 95%, at least 97%, or at least 99% sequence identity to one or more of SEQ ID NOs: 31-35 and 42-71.

9. The dimeric fusion protein according to claim 1, wherein the first polypeptide, the second polypeptide, or both comprise a GLP-1 analogue-Fc fusion protein comprising the amino acid sequence of SEQ ID NO: 37, an amino acid sequence having one, two, three, four or five, amino acid substitutions, additions, or deletions when compared to SEQ ID NO: 37 or an amino acid sequence having at least 80%, at least 85%, at least 90%, at least 95%, at least 97%, or at least 99% sequence identity to SEQ ID NO: 37.

10. The dimeric fusion protein according to claim 1, wherein the first polypeptide, the second polypeptide, or both comprise a GIP analogue-Fc fusion protein comprising the amino acid sequence of SEQ ID No. 36 or an amino acid sequence having one, two, three, four or five, amino acid substitutions, additions, or deletions when compared to SEQ ID NO: 36 or an amino acid sequence having at least 80%, at least 85%, at least 90%, at least 95%, at least 97%, or at least 99% sequence identity to SEQ ID NO: 36.

11. The dimeric fusion protein according to claim 1, wherein the first polypeptide, the second polypeptide, or both is encoded by the polynucleotide SEQ ID NO: 73 or SEQ ID NO: 75, encoded by a polynucleotide having one, two, three, four or five, amino acid substitutions, additions, or deletions when compared to SEQ ID NO: 73 or SEQ ID NO: 75 or encoded by a polynucleotide having at least 80%, at least 85%, at least 90%, at least 95%, at least 97%, or at least 99% sequence identity to SEQ ID NO: 73 or SEQ ID NO: 75.

12. The dimeric fusion protein according to claim 1, wherein the first polypeptide, the second polypeptide, or both comprise SEQ ID NO: 74 or SEQ ID NO: 76, an amino acid sequence having one, two, three, four or five, amino acid substitutions, additions, or deletions when compared to SEQ ID NO: 74 or SEQ ID NO: 76 or an amino acid sequence having at least 80%, at least 85%, at least 90%, at least 95%, at least 97%, or at least 99% sequence identity to SEQ ID NO: 74 or SEQ ID NO: 76.

13. The dimeric fusion protein according to claim 1, wherein an imidazolic group is attached to the N-terminal of P1 and/or P2.

14. The dimeric fusion protein according to claim 1, wherein the fusion protein is a homodimer.

15. The dimeric fusion protein according to claim 1, wherein the fusion protein is a heterodimer.

16. The dimeric fusion protein according to claim 1, wherein at least one of the two peptides comprises at least one unnatural amino acid; wherein the peptide containing unnatural amino acid is chemically synthesized; wherein the N-terminal of the recombinant Fc analogue is Cys; wherein the peptide is fused to the recombinant Fc analogue through native chemical ligation.

17. The dimeric fusion protein according to claim 1, wherein at least one of the two peptides comprises at least one unnatural amino acid and the peptide containing the unnatural amino acid is chemically synthesized; and the chemically synthesized peptide comprises an aldehyde group; and the N-terminal of the recombinant Fc analogue is Cys; and the peptide is conjugated site-specifically to the N-terminal of the recombinant Fc analogue through thiazolidine formation.

18. A pharmaceutical composition comprising a dimeric fusion protein as defined in claim 1.

19. A method of treating a disorder in an individual, the method comprising administering to the individual in need thereof a pharmaceutical composition as defined in claim 18.

20. The method according to claim 19, wherein the disorder is diabetes, obesity, inducement of weight loss in an overweight individual or steatosis.


SEQUENCE LISTING (76 sequences)

SEQ ID NO: 1 - protein, 5 aa, Artificial Sequence
  Linker Sequence
GGGGS

SEQ ID NO: 2 - protein, 180 aa, Homo sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 3 - protein, 37 aa, homo sapiens
HDEFERHAEGTFTSDVSSYLEGQAAKEFIAWLVKGRG

SEQ ID NO: 4 - protein, 37 aa, homo sapiens
HDEFERHAEGTFTSDVSSYLEGQVAKEFIAWLVKGRG

SEQ ID NO: 5 - protein, 31 aa, homo sapiens
HAEGTFTSDVSSYLEGQAAKEFIAWLVKGRG

SEQ ID NO: 6 - protein, 31 aa, homo sapiens
HAEGTFTSDVSSYLEGQVAKEFIAWLVKGRG

SEQ ID NO: 7 - protein, 30 aa, homo sapiens
HAEGTFTSDVSSYLEGQAAKEFIAWLVKGR

SEQ ID NO: 8 - protein, 30 aa, homo sapiens
HAEGTFTSDVSSYLEGQVAKEFIAWLVKGR

SEQ ID NO: 9 - protein, 42 aa, homo sapiens
YAEGTFISDYSIAMDKIHQQDFVNWLLAQKGKKNDWKHNITQ

SEQ ID NO: 10 - protein, 39 aa, homo sapiens
HSDGTFTSDLSKQMEEEAVRLFIEWLKNGGPSSGAPPPS

SEQ ID NO: 11 - protein, 39 aa, homo sapiens
HGEGTFTSDLSKQMEEEAVRLFIEWLKNGGPSSGAPPPS

SEQ ID NO: 12 - protein, 29 aa, homo sapiens
HSQGTFTSDYSKYLDSRRAQDFVQWLMNT

SEQ ID NO: 13 - protein, 37 aa, homo sapiens
HSQGTFTSDYSKYLDSRRAQDFVQWLMNTKRNRNNIA

SEQ ID NO: 14 - protein, 31 aa, artificial sequence
  GLP-1 analogue
HXEGTFTSDVSSYLEEQAALEFIAWLVKGGG

SEQ ID NO: 15 - protein, 31 aa, artificial sequence
  GLP-1 Analogue
HXEGTFTSDVSSYLEEQAALEFIAWLKNGGG

SEQ ID NO: 16 - protein, 31 aa, artificial sequence
  GLP-1 Analogue
HXEGTFTSDVSSYLEEQAALEFIAWLVLGGP

SEQ ID NO: 17 - protein, 31 aa, artificial sequence
  GLP-1 analogue
HXEGTFTSAVSSYLEEQAALEFIAWLKNGGP

SEQ ID NO: 18 - protein, 30 aa, artificial sequence
  GLP-1 Analogue
HXEGTFTSDVSSYLEEQAALEFIAWLKNGG

SEQ ID NO: 19 - protein, 30 aa, artificial sequence; identical to SEQ ID NO: 18
  GLP-1 Analogue

SEQ ID NO: 20 - protein, 42 aa, artificial sequence
  GIP Analogue
YXEGTFISDYSIAMDKIHQQDFVNWLLAQKGKKNDWKHNITQ

SEQ ID NO: 21 - protein, 30 aa, artificial sequence
  GIP Analogue
YXEGTFISDYSIAMDKIHQQDFVNWLLAQK

SEQ ID NO: 22 - protein, 40 aa, artificial sequence
  Exendin analogue
HSDGTFITSDLSKQMEEEAVRLFIEWLKNGGPSSGAPPPS

SEQ ID NO: 23 - protein, 29 aa, artificial sequence
  oxyntomodulin analogue
HXQGTFTSDYSKYLDEKRAKEFVQWLMNT

SEQ ID NO: 24 - protein, 29 aa, artificial sequence
  oxyntomodulin analogue
HXQGTFTSDYSKYLDEKRAKEFVQWLMNT

SEQ ID NO: 25 - protein, 29 aa, artificial sequence
  oxyntomodulin analogue
HXQGTFTSDYSKYLDEQAAKEFICWLMNT

SEQ ID NO: 26 - protein, 29 aa, artificial sequence
  oxyntomodulin analogue
HXQGTFTSDYSKYLDEKRAKEFVQWLMNT

SEQ ID NO: 27 - protein, 29 aa, artificial sequence
  oxyntomodulin analogue
XXQGTFTSDYSKYLDEKRAKEFVQWLMNT

SEQ ID NO: 28 - protein, 29 aa, artificial sequence
  oxyntomodulin analogue
HXQGTFTSDYAKYLDEKRAKEFVQWLMNT

SEQ ID NO: 29 - protein, 37 aa, artificial sequence; identical to SEQ ID NO: 13
  oxyntomodulin analogue

SEQ ID NO: 30 - protein, 29 aa, artificial sequence
  oxyntomodulin analogue
YXQGTFTSDYSKYLDEKRAKEFVQWLMNT

SEQ ID NO: 31 - protein, 327 aa, homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 330 aa, homo sapiens [sequence omitted]

SEQ ID NO: 33 - protein, 326 aa, homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 34 - protein, 452 aa, homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 35 - protein, 230 aa, artificial sequence
  Variant Fc
AESKYGPPCPPCPAPXXXGGPSVFLFPPKPKDXLXIXRXPEVTCVVVDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFXLYSXLTVDKSRWQEGNVFSCSVXHEALHXHYTQKSLSLSLGX

SEQ ID NO: 36 - protein, 275 aa, artificial sequence
  GIP analogue Fc Fusion Protein
YGEGTFISDYSIAMDKIHQQDFVNWLLAQKGGGGGSGGGGGGGGGSAESKYGPPCPPCPAPEAAGGPSVFLFPPKPKDXLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFXLYSXLTVDKSRWQEGNVFSCSVXHEALHNHYTQKSLSLSLG

SEQ ID NO: 37 - protein, 275 aa, artificial sequence
  GLP-1 analogue Fc Fusion Peptide
HGEGTFTSDVSSYLEEQAAKEFIAWLVKGGGGGGGSGGGGGGGGGSAESKYGPPCPPCPAPEAAGGPSVFLFPPKPKDXLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFXLYSXLTVDKSRWQEGNVFSCSVXHEALHNHYTQKSLSLSLG

SEQ ID NO: 38 - protein, 275 aa, artificial sequence
  GLP-1 containing fusion peptide
HGEGTFTSDVSSYLEEQAAKEFIAWLVKGGGGGGGSGGGGGGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVLHEALHNHYTQKSLSLSLG

SEQ ID NO: 39 - protein, 275 aa, artificial sequence
  GIP analogue containing fusion peptide
YGEGTFISDYSIAMDKIHQQDFVNWLLAQKGGGGGSGGGGGGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 40 - protein, 229 aa, artificial sequence
  Fc region
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVLHEALHNHYTQKSLSLSLG

SEQ ID NO: 41 - protein, 8 aa, homo sapiens
KRNRNNIA

SEQ ID NO: 42 - protein, 98 aa, homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 43 - protein, 110 aa, homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 44 - protein, 107 aa, homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 208 aa, artificial sequence
  IgG4 Ch1 and CH2, no hinge
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 46 - protein, 205 aa, artificial sequence
  IgG4 CH1 and CH3, no hinge
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 217 aa, artificial sequence
  IgG4 CH2 and CH3, no hinge
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 98 aa, homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 49 - protein, 113 aa, homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 50 - protein, 107 aa, homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 211 aa, artificial sequence
  IgG1 CH1 and CH2 (no hinge)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 52 - protein, 205 aa, artificial sequence
  IgG1 CH1 and CH3 (no hinge)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 220 aa, artificial sequence
  IgG1 CH2 and CH3 (no hinge)
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 98 aa, homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 55 - protein, 109 aa, homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 56 - protein, 107 aa, homo sapiens
GQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 57 - protein, 207 aa, artificial sequence
  IgG2 CH1 and CH2 no hinge
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 58 - protein, 205 aa, artificial sequence
  IgG2 CH1 and CH3 no hinge
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 59 - protein, 216 aa, artificial sequence
  IgG2 CH2 and CH3 no hinge
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 60 - protein, 105 aa, homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPV

SEQ ID NO: 61 - protein, 112 aa, homo sapiens
IAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPD

SEQ ID NO: 62 - protein, 106 aa, homo sapiens
QDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKG

SEQ ID NO: 63 - protein, 129 aa, homo sapiens
VALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 64 - protein, 217 aa, artificial sequence
  IgM CH1 and CH2
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPD

SEQ ID NO: 65 - protein, 211 aa, artificial sequence
  IgM CH1 and CH3
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKG

SEQ ID NO: 66 - protein, 234 aa, artificial sequence
  IgM CH1 and CH4
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 67 - protein, 340 aa, artificial sequence
  IgM CH1-CH3-CH4
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 68 - protein, 346 aa, artificial sequence
  IgM CH1-CH2-CH4
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 69 - protein, 218 aa, artificial sequence
  IgM CH2-CH3
IAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKG

SEQ ID NO: 70 - protein, 241 aa, artificial sequence
  IgM CH2-CH4
IAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 71 - protein, 346 aa, artificial sequence; identical to SEQ ID NO: 68
  IgM CH2-CH3-CH4

SEQ ID NO: 72 - protein, 129 aa, artificial sequence; identical to SEQ ID NO: 63
  IgM CH3-CH4

SEQ ID NO: 73 - DNA, 908 bp, Artificial Sequence
  DNA encoding the fusion protein ASKBH2
aagcttgccgccaccatggaacgacattggattttccttttcctcttctctgtcaccgccggagtgcatagccacggagagggaaccttcaccagcgacgtgtcctcctacctggaagaacaggccgctaaggagtttatcgcgtggttggtcaagggtggaggcggcgggggagggtccggcggcggaggctcaggcggagggggctccgcagagtcaaaatatgggccgccctgcccgccgtgcccagccccggaggccgcgggcggtccgagcgtgtttttgttcccgcctaaaccgaaggaccagctgatgatttcccgcactcctgaagtcacctgtgtggtggtggacgtgtcacaagaggaccccgaagtgcagttcaattggtacgtcgacggagtggaagtgcacaacgccaagactaagcctcgcgaggagcagttcaactccacgtaccgggtcgtgtcagtgcttactgtgctgcaccaggattggctgaacggaaaggagtacaagtgcaaagtgtccaacaaggggctcccatcgtcgatcgaaaagactatctccaaggccaagggtcaacctcgggagccacaagtctacaccctgcccccgagccaggaagagatgaccaagaaccaagtgtccctgacatgtctcgtgaaggggttctacccctccgacatcgcggtggaatgggaatcgaacggacagcccgaaaacaactacaagaccaccccccctgtcctggatagcgatggcagcttcttcctgtactcgagactcaccgtggacaagtcgcggtggcaggagggcaacgtgttcagctgctccgtgctgcacgaagccctgcacaaccactacacccaaaagtccctgtccctgagcctgggatagttaattaa

SEQ ID NO: 74 - protein, 294 aa, Artificial Sequence
  Fusion protein ASKBH2
MERHWIFLFLFSVTAGVHSHGEGTFTSDVSSYLEEQAAKEFIAWLVKGGGGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 75 - DNA, 908 bp, Artificial Sequence
  DNA encoding the fusion protein ASKBH3
aagcttgccgccaccatggaacgacattggattttccttttcctcttctctgtcaccgccggagtgcatagccacggagagggaaccttcaccagcgacgtgtcctcctacctggaagaacaggccgctaaggagtttatcgcgtggttggtcaagggtggaggcggcgggggagggtccggcggcggaggctcaggcggagggggctccgcagagtcaaaatatgggccgccctgcccgccgtgcccagccccggaggccgcgggcggtccgagcgtgtttttgttcccgcctaaaccgaaggacactctgatgatttcccgcactcctgaagtcacctgtgtggtggtggacgtgtcacaagaggaccccgaagtgcagttcaattggtacgtcgacggagtggaagtgcacaacgccaagactaagcctcgcgaggagcagttcaactccacgtaccgggtcgtgtcagtgcttactgtgctgcaccaggattggctgaacggaaaggagtacaagtgcaaagtgtccaacaaggggctcccatcgtcgatcgaaaagactatctccaaggccaagggtcaacctcgggagccacaagtctacaccctgcccccgagccaggaagagatgaccaagaaccaagtgtccctgacatgtctcgtgaaggggttctacccctccgacatcgcggtggaatgggaatcgaacggacagcccgaaaacaactacaagaccaccccccctgtcctggatagcgatggcagcttcttcctgtactcgaagctcaccgtggacaagtcgcggtggcaggagggcaacgtgttcagctgctccgtgctgcacgaagccctgcacaaccactacacccaaaagtccctgtccctgagcctgggatagttaattaa

SEQ ID NO: 76 - protein, 294 aa, Artificial Sequence
  Fusion protein ASKBH3
MERHWIFLFLFSVTAGVHSHGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVLHEALHNHYTQKSLSLSLG